(19) United States
(12) Patent Application Publication
(10) Pub. No.: US 2026/0150819 A1
(43) Pub. Date: Jun. 4, 2026

(54) NANOS KNOCK-OUT THAT ABLATES GERMLINE CELLS

(71) Applicants: Washington State University, Pullman, WA (US); University of Maryland, College Park, College Park, MD (US); The University Court of the University of Edinburgh, Edinburgh (GB)

(72) Inventors: Jon Michael Oatley, Pullman, WA (US); Christopher Bruce Alexander Whitelaw, Biggar (GB); Simon Geoffrey Lillico, Midlothian (GB); Bhanu Prakash Telugu, College Park, MD (US)

(73) Assignees: Washington State University, Pullman, WA (US); University of Maryland, College Park, College Park, MD (US); The University Court of the University of Edinburgh, Edinburgh (GB)

(21) Appl. No.: 19/387,982

(22) Filed: Nov. 13, 2025

Related U.S. Application Data
(63) Continuation-in-part of application No. 15/325,777, now abandoned.
(60) Provisional application No. 62/023,996, filed on Jul. 14, 2014.

(51) Int. Cl. A01K 67/0276; C12N 9/22; C12N 15/11; C12N 15/86

(52) U.S. Cl. CPC A01K 67/0276; C12N 9/226; C12N 15/111; C12N 15/86; A01K 2217/075; A01K 2227/101; C12N 2310/20

(57) ABSTRACT

The present disclosure provides bovine animals and methods to create recipient bovine animals for spermatogenic stem cell transplantation/complementation through modulation of the NANOS2 gene. In one embodiment genome editing was used to create bovine animals with insertions or deletions (indels) that inactivate or otherwise modulate NANOS2 gene activity so that resulting male bovines lack functional germ cells yet retain functional testicular somatic cells, and bovine females are fertile. These bovine males can then be transplanted or complemented with donor cells capable of giving rise to cells of the spermatogenic lineage, or spermatogenic stem cells and used for breeding.

### CROSS-REFERENCES TO RELATED APPLICATIONS

This application is a continuation-in-part of U.S. patent application Ser. No. 15/325,777, filed Jan. 12, 2017, which is the U.S. National Stage of International Application No. PCT/US2015/040379, filed Jul. 14, 2015, which claims priority to U.S. Provisional Application No. 62/023,996 filed on Jul. 14, 2014, the disclosures of which are incorporated herein by reference in their entirety.

### STATEMENT REGARDING SEQUENCE LISTING

The Sequence Listing XML associated with this application is provided in XML format and is hereby incorporated by reference into the specification. The name of the XML file containing the sequence listing is 4102-P13US.CIP_Seq_List_20250514.xml. The XML file is 109,598 bytes; was created on May 14, 2025; and is being submitted electronically via Patent Center with the filing of the specification.

### FIELD OF THE INVENTION

The present invention relates to a genetically edited non-human livestock animal. The methods of the present invention provide for modified NANOS genes in animals so that males lack germline cells while females are fertile. The resultant male animals are then available for spermatogenic stem cell transplantation or complementation and use in breeding programs.

### BACKGROUND TO THE INVENTION

Genetic gain in livestock can be described as the improvement in production characteristics within a population from generation-to-generation as a result of selective breeding. Exploiting this principle is an important aspect for food animal production to enhance growth efficiency, animal health, and product quality for the consumer while also reducing environmental impact. In livestock production, the majority of genetic gain is made via selective breeding of desirable sires. Thus, expanding the availability of sperm from individual sires can greatly impact food animal production on a global scale. Artificial insemination (AI) methodology has been exploited in commercial livestock production to improve production characteristics worldwide. Despite advances, expansive use of elite boars and bulls for AI in the livestock breeding industry has been hampered due to limitations in the absolute number of sperm that can be collected from an individual. For boars, ejaculates are collected 1-2 times a week and a single ejaculate yields approximately 20 AI doses. Each sow is inseminated 2 to 3 times during a given estrous cycle. Thus, less than 20 sows can be bred each week with sperm from a desirable sire. Thus, novel approaches for expanding the output and availability of gametes from desirable sires and preserving the germline are of significant need.

Spermatogenesis produces millions of sperm daily and the foundation for such vast numbers is provided by the actions of spermatogenic stem cells (SSCs). One of the unique properties of SSCs is their ability to colonize developing testes during embryogenesis and following transplantation or complementation in postnatal life to generate spermatogenesis. An SSC transplantation methodology has been developed for rodent models and adaptation of this approach to pigs would provide an effective breeding tool for expanding and preserving the germline and genetic merit of individual sires. Critical aspects for applying SSC transplantation methodology are: 1) production of recipient animals that lack an endogenous germline, but possess intact support cell populations (i.e., Sertoli and Leydig cells); 2) expansion of the relatively rare donor SSCs in vitro to generate optimal numbers for successful transplantation into several recipient males; and 3) injection of SSCs into recipient testis.

The efficiency of donor SSC colonization is influenced by the environment of recipient testes. Elimination of endogenous germ cells is critical for accessibility of donor SSCs to engraft within seminiferous tubules of recipient testes. In addition, spermatogenesis is regulated by intimate interaction between germ cells and testis support cell populations including Sertoli and Leydig cells. Thus, health of somatic cells at the time of transplantation impacts the success of donor SSC colonization. For rodents, treating adult males with chemotoxic drugs, notably the alkylating agent busulfan and localized testicular irradiation have each been used to effectively prepare recipients for SSC transplantation. While both treatments result in depletion of endogenous germ cells and donor SSCs are able to engraft, the function of somatic support cells is often negatively impacted, and some endogenous germ cells always remain leading to regeneration of a mix of donor and endogenous spermatogenesis. In mice, the greatest success of SSC transplantation involves the use of recipients that are sterile due to inactivation of genes required for germ cell survival at the earliest stages of spermatogenesis. Partial ablation of spermatogenesis in which the spermatogonial population persists is not effective for preparing recipients. In such a case, chemotoxic drugs must still be used to eliminate the persisting spermatogonia thereby opening niches for donor SSCs to engraft. Males in which survival of primordial germ cells (PGCs), gonocytes, or SSCs is compromised provide an ideal recipient.

For pigs and other large domestic animals, treatment with chemotoxic drugs to prepare recipient males is not feasible due to the requirement of high dosage of drugs for complete elimination of germ cells. These treatments often produce unintended consequences of toxicity on bone marrow stem cells and other tissue-specific stem cells. Additionally, feces and urine would need to be collected as a bio-hazardous waste. Local testicular irradiation is a potential alternative that overcomes the limitations of chemotoxic drug treatment; however, the dose of irradiation needs to be precisely controlled, and the procedure inflicts damage on the supporting cells, including Leydig cells thereby negatively affecting the generation of donor-derived spermatogenesis. Ideal recipients are males lacking an endogenous germline due to a genetic deficiency that leaves the somatic support cell population functionally intact.

As can be seen, there is a need in the art for animals where the male has no germ cells but retains functional somatic cells and is thus eligible for SSC transplantation or complementation, while ideally females are fertile.

### SUMMARY OF THE INVENTION

The present disclosure provides animals and methods for spermatogenic stem cell transplantation by creating recipient bovine animals that have modulated NANOS2 expression. The bovine animals have inactivated or otherwise modulated NANOS2 gene activity resulting in males which lack functional germ cells yet retain functional somatic cells, and females which are fertile. These bovine animals can be created using any of a number of protocols such as knock-out technology or gene-editing.

Thus, an embodiment of the disclosure is a genetically edited or modified bovine animal comprising a genome with inactivation of a NANOS2 gene selective for germline cell function.

Yet another embodiment of the disclosure is a process of making a bovine animal comprising introducing to a bovine animal cell or bovine embryo, an agent that specifically binds to a chromosomal target site of the cell and causes a double-stranded DNA break or otherwise inactivates a NANOS2 gene therein using gene-editing methods such as the Clustered Regularly Interspaced Short Palindromic Repeats (CRISPR)/Cas system, Transcription Activator-Like Effector Nucleases (TALENs), Zinc Finger Nucleases (ZFN), or recombinase fusion proteins.

Yet another embodiment of the disclosure is a process of producing a male sperm donor for bovine breeding with a desired spermatogenic stem cell genetic component comprising; collecting donor spermatogenic stem cells (SSCs) from a desired male donor, proliferating SSCs in vitro, and thereafter transplanting donor SSCs to a NANOS2^{-/-} male so that spermatogenic colonies are generated and persist for a long period of time with the donor germline cells.

Yet another embodiment of the disclosure includes the production of bovine animals comprising natural mating and/or artificial insemination of female a bovine animal with donor sperm from the recipient NANAOS2^{-/-} male.

Also described herein is the use of one or more particular NANOS2 loci in tandem with a polypeptide capable of affecting cleavage and/or integration of specific nucleic acid sequences within the NANOS2 loci. Examples of the use of NANOS2 loci in tandem with a polypeptide capable of effecting cleavage and/or integration of the NANOS2 loci include a polypeptide selected from the group consisting of zinc finger proteins, meganucleases, TAL domains, TALENs, RNA-guided CRISPR/Cas recombinases, leucine zippers, and others known to those in the art. Particular examples include a chimeric (“fusion”) protein comprising a site-specific DNA binding domain polypeptide and cleavage domain polypeptide (e.g., a nuclease), such as a ZFN protein comprising a zinc-finger polypeptide and a FokI nuclease polypeptide. In certain aspects described herein are polypeptides comprising a DNA-binding domain that specifically binds to a NANOS2 gene. In some embodiments such a polypeptide may also comprise a nuclease (cleavage) domain or half-domain (e.g., a ZFN, a recombinase, a transposase, or a homing endonuclease, including a homing endonuclease with a modified DNA-binding domain, TAL domains, TALENs, RNA-guided CRISPR/Cas), and/or a ligase domain, such that the polypeptide can induce a targeted double-stranded break, and/or facilitate recombination of a nucleic acid of interest at the site of the break. In particular embodiments, a DNA-binding domain that targets a NANOS2 locus may be a DNA-cleaving functional domain. The foregoing polypeptides can be used in some embodiments to introduce an exogenous nucleic acid into the genome of a host organism (e.g., a bovine animal species) at one or more NANOS2 loci. In certain embodiments, the DNA-binding domains comprise a zinc finger protein with one or more zinc fingers (e.g., 2, 3, 4, 5, 6, 7, 8, 9 or more zinc fingers), which is engineered (non-naturally occurring) to bind to any sequence within a NANOS2 gene. Any of the zinc finger proteins described herein can bind to a target site within the coding sequence of the target gene or within adjacent sequences (e.g., promoter or other expression elements). In certain embodiments, the zinc finger protein binds to a target site in a NANOS2 gene, for example, approximately 20 bases in exon 1.

Further embodiments will become evident from the detailed description which follows.

### DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1C3. FIGS. 1A and 1B depict plasmids comprising guide RNA sequences for NANOS2. FIG. 1A is a map of the pSpCas9(BB)-2A-GFP (PX458) plasmid. FIG. 1B shows the sequence annotations pX458 partial sequence (SEQ ID NO: 7); hU6 sequence (SEQ ID NO:8), gRNA sequence (SEQ ID NO:9), and terminal sequence (SEQ ID NO: 10). FIGS. 1C1-1C3 show the entire construct sequence (SEQ ID NO: 11).

FIGS. 2A and 2B are gels of PCR products from genomic DNA post transfection and showed substantial differences in the efficiencies with which sgRNAs were able to induce NHEJ formation at their target site as indicated by the presence of T7 endonuclease digestion products (short right facing arrows).

FIG. 3 shows deletions generated by the use of different combinations of CRISPRs. Photo is a gel of genomic DNA of indels amplified with primers oSL868 (SEQ ID NO: 15) and oSL87 (SEQ ID NO:25) of the six plasmid combinations.

FIG. 4 shows the sequences of the bovine indels compared to wild-type. pSL32 & pSL38: WT (SEQ ID NO:55), Clone 1 (SEQ ID NO:56); CLONE 2 (SEQ ID NO: 56); CLONE 3 (SEQ ID NO:57); CLONE 4 (SEQ ID NO:57); CLONE 5 (SEQ IDNO: 58).

pSL32 & pSL39: WT (SEQ ID NO:55), Clone 1 (SEQ ID NO:59); CLONE 2 (SEQ ID NO: 60); CLONE 3 (SEQ ID NO:61); CLONE 4 (SEQ ID NO:62); CLONE 5 (SEQ ID NO: 60).

pSL32 & pSL42 WT (SEQ ID NO:55), Clone 1 (SEQ ID NO:63); CLONE 2 (SEQ ID NO: 64); CLONE 3 (SEQ ID NO:65); CLONE 4 (SEQ ID NO:66); CLONE 5 (SEQ ID NO: 67).

pSL33 & pSL38 WT (SEQ ID NO:55), Clone 1 (SEQ ID NO:68); CLONE 2 (SEQ ID NO: 68); CLONE 3 (SEQ ID NO:68); CLONE 4 (SEQ ID NO:68); CLONE 5 (SEQ ID NO: 68).

pSL33 & pSL39 WT (SEQ ID NO:55), Clone 1 (SEQ ID NO:69); CLONE 2 (SEQ ID NO: 70); CLONE 3 (SEQ ID NO:71); CLONE 4 (SEQ ID NO:72); CLONE 5 (SEQ ID NO: 73).

pSL33 & pSL42 WT (SEQ ID NO:55), Clone 1 (SEQ ID NO:74); CLONE 2 (SEQ ID NO: 75); CLONE 3 (SEQ ID NO:76); CLONE 4 (SEQ ID NO:751); CLONE 5 (SEQ ID NO: 77).

FIG. 5 shows the bovine NANOS2 sequence with the designed guides and amplification primers annotated. Entire nucleotide sequence (SEQ ID NO:12); NANOS2 nt CDS (SEQ ID NO:14); First 14 amino acids of NANOS2 (SEQ ID NO:13); oSL868 (SEQ ID NO: 15); pSL36 or 37 (SEQ ID NO:16); pSL34 or 35 (SEQ ID NO:17); pSL32 or 33 (SEQ ID NO:18); pSL38 or 39 (SEQ ID NO:19); pSL39 or 40 (SEQ ID NO:20); pSL41 or 42 (SEQ ID NO:21); pSL43 or 44 (SEQ ID NO:22); pSL45 or 46 (SEQ ID NO:23); pSL47 or 48 (SEQ ID NO:24); oSL87 (SEQ ID NO:25).

FIG. 6 is a schematic of the CRISPR/Cas9 design for targeted knock-out of the NANOS2 gene in bovine embryos. Blue font denotes exon sequence. Purple font denotes primer sequences for amplicon-based genotyping. Boxed sequences designate the guide RNAs used for CRISPR/Cas9 editing.

FIGS. 7A-7C depict the generation of NANOS2 knock-out (KO) male and female cattle via CRISPR-Cas9 gene editing. FIG. 7A shows representative images of an adult male (bull) and an adult female (heifer) that were generated from embryos treated with CRISPR/Cas9 gene editing components targeting the NANOS2 gene. FIG. 7B shows PCR amplicon based genotyping analysis for NANOS2 alleles in the bull and heifer shown in FIG. 7A. Genomic DNA was isolated from blood cells and hair follicle cells for analysis. Genomic DNA from a cow that was not subjected to CRISPR/Cas9 editing was used as a wild-type (WT) control. The reduced size amplicons in the NANOS2 CRISPR/Cas9 animals demonstrated deletion mutations in the genes. FIG. 7C shows the outcomes of DNA sequencing analysis for mutated NANOS2 alleles in the NANOS2 CRISPR/Cas9 bull. The animal possessed bi-allelic inactivating deletion mutations demonstrating a true NANOS2 knock-out genotype. Wild-type testis Allele 1 (SEQ ID NO:99; NANOS2 CRISPR/Cas9 Bull-Hair Follicle-Allele 1 (SEQ ID NO:100) and Allele 2 (SEQ ID NO: 101).

FIGS. 8A-8C depict the generation of NANOS2 knock-out male (bull) bovine animals via CRISPR/Cas9 gene editing. FIG. 8A shows representative images of two NANOS2 knock-out bulls that were generated by CRISPR/Cas9 gene editing. FIG. 8B depict images of agarose gels to visualize PCR genotyping for the NANOS2 gene using genomic DNA isolated from blood and hair cells of CRISPR/Cas9 edited Bulls #1 and #2 and a wild-type (WT) non-edited bull. Lanes labeled as N/A are for different animals and not applicable to this example. FIG. 8C shows the outcome of tracking of INDELs by decomposition (TIDE) analysis for DNA sequencing of the NANOS2 gene in CRISPR-Cas9 gene edited bulls #1 and #2. Position 0 on the x-axis denotes no mutations whereas sequences detected that fall to the left of 0 on the x-axis are deletion mutations. The outcome demonstrated that Bull #1 possessed NANOS2 deletion mutations of 16 base pairs, 22 base pairs, 37 base pairs, and 313 base pairs; and Bull #2 possessed NANOS2 deletion mutations of 370 base pairs and 510 base pairs. All the detected mutations are frameshifting or removed most of, if not, all the NANOS2 coding sequence. All mutations yield a genetic knock-out condition.

FIG. 9 shows histological assessment of testicular tissue from an E0 NANOS2 bull at four months of age. The top panels show hemotoxylin and eosin staining of testicular cross-sections from a non-CRISPR/Cas9 edited wild-type (WT) bull at four months of age and E0 NANOS2 CRISPR/Cas9 knock-out bull at four months of age. Arrows indicate germ cells that are present in the WT sample which cannot be observed in the sample from the NANOS2 knockout bull. The bottom panels show images of immunostaining from the germ cell biomarker DDX4 in cross-sections of testicular tissue from a non-CRISPR/Cas9 edited wild-type (WT) bull at four months of age and E0 NANOS2 CRISPR/Cas9 knock-out bull at four months of age. Absence of dark staining in the NANOS2 knock-out sample demonstrated endogenous germline ablation.

FIGS. 10A-10C depict the outcomes of donor spermatogenic stem cell transplantation into the testes of an endogenous germline ablated NANOS2 knock-out bull. FIG. 10A is an image of sperm in the ejaculate of a NANOS2 knock-out bull transplanted with donor spermatogenic stem cells. FIG. 10B is an image of morula and blastocyst stage embryos generated by in vitro fertilization with sperm collected from the spermatogenic stem cell transplanted NANOS2 knock-out bull and eggs of a wild-type cow. The NANOS2 knock-out recipient bull was heterozygous for horned and polled alleles (H/P). The spermatogenic stem cell donor was homozygous for horned alleles (H/H). The egg donor cow was homozygous for polled alleles (P/P). FIG. 10C is a representative image of PCR amplicon-based genotyping of six in vitro fertilized embryos for horned and polled alleles. All (100%) of embryos genotyped as heterozygous for horned and polled alleles (H/P) demonstrating that the sperm were of spermatogenic stem cell donor origin.

FIGS. 11A-11C show pictures of four NANOS2 knock-out heifers that were generated by CRISPR/Cas9 gene editing (FIG. 11A). Image of agarose gels to visualize PCR genotyping for the NANOS2 gene using genomic DNA isolated from blood cells (Heifer #1) and/or hair cells (Heifers #1-4) of CRISPR/Cas9 edited heifers and a wild-type (WT) non-edited heifer (FIG. 11B). Outcome of tracking of INDELs by decomposition (TIDE) analysis for DNA sequencing of the NANOS2 gene in CRISPR/Cas9 gene edited heifers #1-4 (FIG. 11C). Position 0 on the x-axis denotes no mutations whereas sequences detected that fall to the left of 0 on the x-axis are deletion mutations. The outcome demonstrates that Heifer #1 possessed NANOS2 deletion mutations of 368 base pairs, and 376 base pairs; Heifer #2 possessed a NANOS2 deletion mutation of 370 base pairs only; Heifer #3 possessed NANOS2 deletion mutations of 450 base pairs and 466 base pairs; and Heifer #4 possessed NANOS2 deletion mutations of 814 base pairs. None of the heifers were found to possess a wild-type NANOS2 allele. All the detected mutations are frameshifting or removed most of, if not, all the NANOS2 coding sequence. All mutations yield a genetic knock-out condition.

FIGS. 12A-12B show a picture of NANOS2^{+/-} heterozygous bull that was generated by breeding a NANOS2^{-/-} knock-out heifer with a wild-type bull (FIG. 12A). Image of an agarose gel to visualize PCR genotyping for the NANOS2 gene using genomic DNA isolated from hair cells of the NANOS2^{+/-} bull and a wild-type (WT) non-edited bull (FIG. 12B). These data are an unequivocal demonstration that NANOS2^{-/-} knock-out female cattle are fertile.

### DETAILED DESCRIPTION OF THE INVENTION

The present methods and genetically edited bovine animals and other livestock now will be described more fully with reference to the accompanying examples. The methods and resultant gene edited bovine animals can be embodied in many different forms and should not be construed as limited to the embodiments set forth in this application; rather, these embodiments are provided so that this disclosure will satisfy applicable legal requirements.

Many modifications and other embodiments of the disclosure will come to mind to one skilled in the art to which these methods and resultant genetically edited bovine animals pertain, having the benefit of the teachings presented in the descriptions and the drawings herein. As a result, it is to be understood that the methods and resultant genetically edited bovine animals are not to be limited to the specific embodiments disclosed and that modifications and other embodiments are intended to be included within the scope of the appended claims. Although specific terms are used in the specification, they are used in a generic and descriptive sense only and not for purposes of limitation.

Units, prefixes, and symbols may be denoted in their SI accepted form. Unless otherwise indicated, nucleic acids are written left to right in 5′ to 3′ orientation; amino acid sequences are written left to right in amino- to carboxy-orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence-based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

The term “conservatively modified variants” applies to both amino acid and nucleic acid sequences. With respect to particular nucleic acid sequences, “conservatively modified variants” refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid.

One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present disclosure is implicit in each described polypeptide sequence and is within the scope of the present disclosure.

As to amino acid sequences, one of ordinary skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made.

Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another: 1) Alanine (A), Serine(S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). See also, Creighton (1984) Proteins W. H. Freeman and Company.

By “encoding” or “encoded”, with respect to a specified nucleic acid, is meant comprising the information for translation into the specified protein. A nucleic acid encoding a protein can comprise intervening sequences (e.g., introns) within translated regions of the nucleic acid, or can lack such intervening non-translated sequences (e.g., as in cDNA). The information by which a protein is encoded is specified by the use of codons. Typically, the amino acid sequence is encoded by the nucleic acid using the “universal” genetic code. When the nucleic acid is prepared or altered synthetically, advantage can be taken of known codon preferences of the intended host where the nucleic acid is to be expressed.

As used herein “full-length sequence” in reference to a specified polynucleotide or its encoded protein means having the entire amino acid sequence of a native (nonsynthetic), endogenous, biologically active form of the specified protein. Methods to determine whether a sequence is full-length are well known in the art including such exemplary techniques as northern or western blots, primer extension, S1 protection, and ribonuclease protection. Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous” in reference to a nucleic acid is a nucleic acid that originates from a foreign or heterologous species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein can originate from a foreign species or, if from the same species, is substantially modified from its original form by deliberate human intervention.

By “host cell” is meant a cell which contains a vector and supports the replication and/or expression of the vector. Host cells can be prokaryotic cells such as E. coli, or eukaryotic cells such as yeast, insect, amphibian, or mammalian cells.

The term “hybridization complex” includes reference to a duplex nucleic acid structure formed by two single-stranded nucleic acid sequences selectively hybridized with each other.

The term “introduced” in the context of inserting a nucleic acid into a cell is equivalent to “transfection” or “transformation” or “transduction,” and includes reference to the incorporation of a nucleic acid into a eukaryotic or prokaryotic cell where the nucleic acid may be incorporated into the genome of the cell (e. g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components that normally accompany or interact with it as found in its naturally occurring environment—the isolated material optionally comprises material not found with the material in its natural environment; or (2) if the material is in its natural environment, the material has been synthetically altered by deliberate human intervention to a composition and/or placed at a location in the cell (e.g., genome or subcellular organelle) not native that material. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “localized within the chromosomal region defined by and including” with respect to particular markers includes reference to a contiguous length of a chromosome delimited by and including the stated markers.

As used herein, “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes of that pair to be followed. A genotype may be defined by the use of one or a plurality of markers.

As used herein, “mutation” includes reference to alterations in the nucleotide sequence of a polynucleotide, such as for example a gene or coding DNA sequence (CDS), compared to the wild-type sequence. The term includes, without limitation, substitutions, insertions, frameshifts, deletions, inversions, translocations, duplications, splice-donor site mutations, point-mutations, or the like.

As used herein, “nucleic acid” includes reference to a deoxyribonucleotide or ribonucleotide polymer in either single- or double-stranded form, and unless otherwise limited, encompasses conservatively modified variants and known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, CA (Berger); Sambrook et al., Molecular Cloning-A Laboratory Manual, 2nded., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary, join two protein coding regions, contiguously and in the same reading frame.

As used herein, “polynucleotide” includes reference to a deoxyribopolynucleotide, ribopolynucleotide, or conservatively modified variants; the term may also refer to analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as, for example, inosine, or modified bases, such as tritylated bases, to name just two examples, are polynucleotides as the term is used herein. It will be appreciated that a great variety of modifications have been made to DNA and RNA that serve many useful purposes known to those of skill in the art.

The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms also apply to conservatively modified variants and to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, the protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not always entirely linear. For instance, polypeptides may be branched as a result of ubiquitization, and they may be circular, with or without branching, generally as a result of posttranslation events, including natural processing events, and events brought about by human manipulation which do not occur naturally. Circular, branched, and branched circular polypeptides can be synthesized by non-translation natural process and by entirely synthetic methods, as well. Further, the present disclosure contemplates the use of both the methionine-containing and the methionine-less amino terminal variants of the protein of the disclosure.

As used herein “promoter” includes reference to a region of DNA upstream from the start of transcription and involved in recognition and binding of RNA polymerase and other proteins to initiate transcription. Examples of promoters under developmental control include promoters that preferentially initiate transcription in certain tissues, such as testes, ovaries, or placenta. Such promoters are referred to as “tissue preferred”. Promoters which initiate transcription only in certain tissue are referred to as “tissue specific”. A “cell type” specific promoter primarily drives expression in certain cell types in one or more organs, for example, germ cells in testes or ovaries. An “inducible” or “repressible” promoter is a promoter which is under environmental control. Examples of environmental conditions that can affect transcription by inducible promoters include stress, and temperature. Tissue specific, tissue preferred, cell type specific and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in an identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to another nucleic acid sequence or other biologics. When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule.

The term “stringent conditions” or “stringent hybridization conditions” includes reference to conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing).

Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Specificity is typically the function of post-hybridization washes, the critical factors being the ionic strength and temperature of the final wash solution. For DNA/DNA hybrids, the thermal melting point (Tm) can be approximated from the equation of Meinkoth and Wahl Anal. Biochem. 138:267-284 (1984):

Tm [° C.]=81.5+16.6 (log M)+0.41(% GC)−0.61(% form)−500/L;

where M is the molarity of monovalent cations, % GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with >90% identity are sought, the Tm can be decreased 10° C. Generally, stringent conditions are selected to be about 5° C. lower than the Tm for the specific sequence and its complement at a defined ionic strength and pH. However, severely stringent conditions can utilize a hybridization step and/or wash at 1 to 4° C. lower than the Tm; moderately stringent conditions can utilize a hybridization step and/or wash at 6 to 10° C. lower than the Tm; low stringency conditions can utilize a hybridization step and/or wash at 11 to 20° C. lower than the Tm. Using the equation, hybridization and wash compositions, and desired Tm, those of ordinary skill will understand that variations in the stringency of hybridization and/or wash solutions are inherently described. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel, et al., Eds., Greene Publishing and Wiley-Interscience, New York (1995).

As used herein, “transgenic” or “genetically edited animal, cell or tissue” includes reference to an animal which includes within its genome a heterologous polynucleotide. Generally, the heterologous polynucleotide is stably integrated within the genome such that the polynucleotide is passed on to successive generations. The heterologous polynucleotide can be integrated into the genome alone or as part of a recombinant expression cassette. “Transgenic” is used herein to include any cell, cell line, tissue, or organ, the genotype of which has been altered by the presence of heterologous nucleic acid including those transgenics or genetically edited initially so altered as well as those created by sexual crosses or asexual propagation from the initial transgenic or genetically edited animal. The term “transgenic” or “genetically edited” as used herein does not encompass the alteration of the genome (chromosomal or extra-chromosomal) by conventional breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which a polynucleotide can be inserted. Vectors are often replicons. Expression vectors permit transcription of a nucleic acid inserted therein.

The following terms are used to describe the sequence relationships between a polynucleotide/polypeptide of the present invention with a reference polynucleotide/polypeptide: (a) “reference sequence”, (b) “comparison window”, (c) “sequence identity”, and (d) “percentage of sequence identity”.

(a) As used herein, “reference sequence” is a defined sequence used as a basis for sequence comparison with a polynucleotide/polypeptide of the present invention. A reference sequence can be a subset or the entirety of a specified sequence, for example, as a segment of a full-length cDNA or gene sequence, or the complete cDNA or gene sequence.

(b) As used herein, a “comparison window” includes reference to a contiguous and specified segment of a polynucleotide/polypeptide sequence, wherein the polynucleotide/polypeptide sequence can be compared to a reference sequence and wherein the portion of the polynucleotide/polypeptide sequence in the comparison window can comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison can be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. USA 85:2444 (1988); and by computerized implementations of these algorithms, including, but not limited to: CLUSTAL in the PC/Gene program by Intelligenetics, Mountain View, California; GAP, BESTFIT, BLAST, FASTA, and TFASTA, and related programs in the GCG Wisconsin Genetics Software Package, Version 10 (available from Accelrys Inc., 9685 Scranton Road, San Diego, California, USA). The CLUSTAL program is well described by Higgins and Sharp, Gene 73:237-244 (1988); Higgins and Sharp, CABIOS 5:151-153 (1989); Corpet et al., Nucl. Acids Res. 16:10881-10890 (1988); Huang et al., CABIOS 8:155-165 (1992), and Pearson et al., Meth. Mol. Biol. 24:307-331 (1994).

The BLAST family of programs that can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol. 215:403-410 (1990); and Altschul et al., Nucl. Acids Res. 25:3389-3402 (1997). Software for performing BLAST analyses is publicly available, for example through the website for the National Center for Biotechnology Information. This algorithm has been thoroughly described in a number of publications. See, e.g., Altschul S F et al., Gapped BLAST and PSI-BLAST: a new generation of protein database search programs, NUCL. ACIDS RES. 25(17):3389-3402 (1997); National Center for Biotechnology Information, THE NCBI HANDBOOK [INTERNET], Chapter 16: The BLAST Sequence Analysis Tool (McEntyre J, Ostell J, eds., 2002). The BLASTP program for amino acid sequences has also been thoroughly described (see Henikoff and Henikoff Proc. Natl. Acad. Sci. USA 89:10915 (1989)).

In addition to calculating percent sequence identity, the BLAST algorithm also performs a statistical analysis of the similarity between two sequences (see, e.g., Karlin and Altschul, Proc. Natl. Acad. Sci. USA 90:5873-5877 (1993)). A number of low-complexity filter programs can be employed to reduce such low-complexity alignments. For example, the SEG (Wooten and Federhen, Comput. Chem. 17:149-163 (1993)) and XNU (Claverie and States, Comput. Chem. 17:191-201 (1993)) low-complexity filters can be employed alone or in combination.

Unless otherwise stated, nucleotide and protein identity/similarity values provided herein are calculated using GAP (GCG Version 10) under default values. GAP (Global Alignment Program) can also be used to compare a polynucleotide or polypeptide of the present invention with a reference sequence. GAP uses the algorithm of Needleman and Wunsch (J. Mol. Biol. 48:443-453, 1970) to find the alignment of two complete sequences that maximize the number of matches and minimize the number of gaps. GAP represents one member of the family of best alignments. There can be many members of this family, but no other member has a better quality. GAP displays four figures of merit for alignments: “Quality,” “Ratio,” “Identity,” and “Similarity.” The “Quality” is the metric maximized in order to align the sequences. “Ratio” is the quality divided by the number of bases in the shorter segment. “Percent Identity” is the percent of the symbols that actually match. “Percent Similarity” is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff, Proc. Natl. Acad. Sci. USA 89:10915 (1989)).

Multiple alignment of the sequences can be performed using the CLUSTAL method of alignment (Higgins and Sharp, CABIOS 5:151-153 (1989)) with the default parameters (GAPPENALTY=10, GAP LENGTH PENALTY=10). Default parameters for pairwise alignments using the CLUSTAL method include KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and one. The scoring of conservative substitutions can be calculated according to the algorithm of Meyers and Miller, Computer Applic. Biol. Sci. 4:11-17 (1988), for example as implemented in the program PC/GENE (Intelligenetics, Mountain View, California, USA).

(d) As used herein, “percentage of sequence identity” means the value determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, “gene editing,” “gene edited,” “genetically edited,” and “gene editing effectors” refer to the use of naturally occurring or artificially engineered nucleases, also referred to as “molecular scissors.” The nucleases create specific double-stranded break (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), the Clustered Regularly Interspaced Short Palindromic Repeats/CAS9 (CRISPR/Cas9) system, and meganuclease re-engineered as homing endonucleases. The terms also include the use of transgenic procedures and techniques, including, for example, where the change is relatively small and/or does not introduce DNA from a foreign species. The terms “genetic manipulation” and “genetically manipulated” include gene editing techniques, as well as and/or in addition to other techniques and processes that alter or modify the nucleotide sequence of a gene or genes or modify or alter the expression of a gene or genes.

As used herein “homing DNA technology” or “homing technology” covers any mechanisms that allow a specified molecule to be targeted to a specified DNA sequence including Zinc Finger (ZF) proteins, Transcription Activator-Like Effectors (TALEs) meganucleases, and the CRISPR/Cas9 system.

The term “livestock animal” includes animals traditionally raised in livestock farming, such as beef cattle, dairy cattle, pigs, sheep, goats, horses, mules, asses, buffalo, and camels. This does not include rats, mice, or other rodents.

As used herein “blastocyst” means an early developmental stage of embryo comprising of inner cell mass (from which an embryo proper arises) and a fluid filled cavity typically surrounded by a single layer of trophoblast cells. “Developmental Biology”, sixth edition, ed. by Scott F. Gilbert, Sinauer Associates, Inc., Publishers, Sunderland, Mass. (2000)

As used herein “Conditional Knock-out” or “Conditional mutation” means when the knock-out or mutation is achieved when certain conditions are met. These conditions include but are not limited to presence of certain inducing agents, recombinases, antibiotics, and certain temperature or salt levels.

The term “Early-stage embryo” means any embryo at embryonic stages between fertilized ovum and blastocyst. Typically, eight-cell stage and morula stage embryos are referred to as early-stage embryos.

“Embryonic germ cells” or “EG cells” means primordial germ cell derived cells which have the potential to differentiate into all the cell types of body and are as amenable to genetic modification as Embryonic stem cells, to the extent that sometimes the distinction between EG cells and ES cells is ignored. “Developmental Biology”, sixth edition, ed. by Scott F. Gilbert, Sinauer Associates, Inc., Publishers, Sunderland, Mass. (2000).

“Embryonic stem cells” or “ES cells” means cultured cells derived from inner cell mass of an early-stage embryo, which are amenable to genetic modification, and which retain their totipotency and can contribute to all organs of the resulting chimeric animal if injected into the host embryo. “Developmental Biology”, sixth edition, ed. by Scott F. Gilbert, Sinauer Associates, Inc., Publishers, Sunderland, Mass. (2000).

As used herein, “Fertilization” means the union of male and female gametes during reproduction resulting in the formation of a zygote, the earliest developmental stage of an embryo. “Foreign cell” means any cell that can be genetically edited or can be derived from a genetically edited cell and that can contribute towards the germ line of a chimeric embryo when injected or aggregated with a donor blastocyst/embryo. This includes, but is not limited to, embryonic stem (ES) cells, teratocarcinoma stem cells, primordial germ cells, and embryonic germ (EG) cells.

The phrase “Genetically edited” means those animals or embryos or cells which have a desired genetic modification such as a knock-out, a knock-in, a conditional, an inducible, a transient or a point mutation(s) of any gene or its regulatory mechanism or a transgenic with foreign or modified gene/s or regulatory sequences, or having undergone genomic modification in any way including but not limited to recombination, chromosomal deletion, addition, translocation, rearrangement or addition, deletion or modification of nucleic acid, protein or any other natural or synthetic molecule or organelle, or cytoplasmic or nuclear transfer, leading to inheritable changes.

“Germ cell development” means the process by which certain cells in the early stage developing embryo differentiate into primordial germ cells.

“Germ cell migration” means the process by which primordial germ cells, after originating in the extraembryonic mesoderm travel back in the embryo through allantois (precursor of umbilical cord) and continue to migrate through adjacent yolk sac, hindgut, and dorsal mesentery to finally reach the genital ridge (developing gonad). “Developmental Biology”, sixth edition, ed. by Scott F. Gilbert, Sinauer Associates, Inc., Publishers, Sunderland, Mass. (2000).

“Germ line cell” means any cell, at any stage of differentiation towards mature gametes, including mature gametes.

As used herein, the term “Knock-in” means replacement of an endogenous gene with a transgene or with same endogenous gene with some structural modification(s) but retaining the transcriptional control of the endogenous gene.

“Knock-out” means disruption of the structure or regulatory mechanism of a gene. Knock-outs may be generated through homologous recombination of targeting vectors, replacement vectors or hit-and-run vectors or random insertion of a gene trap vector resulting in complete, partial or conditional loss of gene function. “Oogenesis” means the process of generation of mature eggs from the primordial germ cells in females.

“Primordial germ cells” means those cells arising early in the embryonic development that give rise to the spermatogenic lineage via a gonocyte intermediate or female germline via an oogonia intermediate.

“Spermatogenesis” means the process of generation of mature sperm from spermatogenic stem cells in males. “Spermatogenic stem cells” comprise any primordial germ cell that is capable of differentiating into a spermatogonia and eventually a sperm cell by the process of spermatogenesis.

“Spermatogenic stem cell” means any cell that has the capacity to generate or regenerate the spermatogenic lineage to produce spermatogenesis.

“Wild-type” means those animals and blastocysts, embryos or cells derived therefrom, which have not been genetically edited and are usually inbred and outbred strains developed from naturally occurring strains. A “binding protein” is a protein that is able to bind to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, and the like) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein.

Zinc finger and TALE binding domains can be “engineered” to bind to a predetermined nucleotide sequence, for example via engineering (altering one or more amino acids) of the recognition helix region of naturally occurring zinc finger or TALE proteins. Therefore, engineered DNA binding proteins (zinc fingers or TALEs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

A “selected” zinc finger protein or TALE is a protein not found in nature whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970 WO 01/88197, WO 02/099084 and U.S. Publication No. 20110301073.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 2007/0218528, 2008/0131962 and 2011/0201055, incorporated herein by reference in their entirety.

Means for generating a double strand DNA break: As used herein, the term “means for generating a double strand DNA break” is intended to invoke the special claiming provisions authorized by Congress in 35 U.S.C. § 112, sixth paragraph. Specifically, a “means for generating a double strand DNA break” refers to a molecular structure that is capable of cleaving both strands of a double-stranded DNA molecule. Such structures include polypeptide domains comprised within many known nuclease proteins, for example, the FokI nuclease domain, the catalytic domain is selected from the group consisting of proteins Mme1, Colicin-E7 (CEA7_ECOLX), Colicin-E9, APFL, EndA, Endo I (END1_EC0LI), Human Endo G (NUCG_HUMAN), Bovine Endo G (NUCG_BOVIN), R.HinP11, 1-Bas-1, 1-Bmo-1, 1-Hmu1, 1-Tev-1, 1-Tev11, 1-Tev111, 1-Twol, R.Msp1, R.Mva1, NucA, NucM, Vvn, Vvn_CLS, Staphylococcal nuclease (NUC_STAAU), Staphylococcal nuclease (NUC_STAHY), Micrococcal nuclease (NUC_SHIFL), Endonuclease yncB, Endodeoxyribonuclease I (ENRN_BPT7), Metnase, Nb.BsrDI, BsrDI A, Nt.BspD61 (R.BspD61 large subunit), ss.BspD61 (R.BspD61 small subunit), R.Ple1, Mly1, Alw1, Mva12691, Bsr1, Bsm1, Nb.BtsCI, Nt.BtsCI, R1.Bts1, R2.Bts1, BbvCI subunit 1, BbvCI subunit 2, BpulOI alpha subunit, BpulOI beta subunit, Bmr1, Bfi1, 1-Cre1, hExo1 (EX01JHUMAN), Yeast Exo1 (EX01_YEAST), E. coli Exo1, Human TREX2, Mouse TREX1, Human TREX1, Bovine TREX1, Rat TREX1, Human DNA2, Yeast DNA2 (DNA2 YEAST).

As used herein, the term “means for repairing a double strand DNA break” is also intended to invoke the special claiming provisions authorized by Congress in 35 U.S.C. sctn.112, sixth paragraph. Specifically, a “means for repairing a double strand DNA break” refers to a molecular structure that is capable of facilitating/catalyzing the joining of the ends of double-stranded DNA molecules, for example, by joining ends generated by cleaving a single double-stranded DNA molecule, or by joining one end generated by cleaving a single double-stranded DNA molecule with the end of an exogenous double-stranded DNA molecule. Such structures include polypeptide domains comprised within many known ligase proteins, for example, Cre recombinase. In some examples, the same molecular structure may serve as both a means for generating a double strand DNA break and a means for repairing a double strand DNA break, where the same structure facilitates both the cleavage and repair of double-stranded DNA molecules (e.g., a Hin recombinase).

The induction of the site specific double stranded breaks in the genome induces the host cell DNA repair pathway which resolves the double stranded break through homology-directed repair (HDR) or non-homologous end-joining (NHEJ) repair. It is possible to have one or more ZFN cut sites on the donor molecule (a single ZFN cut site to linearize the entire donor molecule, 2 of the same ZFN sites to release a smaller donor DNA fragment or 2 different ZFN sites to release a fragment from the donor and a corresponding fragment from the host genomic DNA (DNA replacement).

Thus, the donor polynucleotide can be DNA or RNA, single-stranded and/or double-stranded and can be introduced into a cell in linear or circular form. See, e.g., U.S. Patent Application Publication Nos. 2010/0047805 and 2011/0207221. In certain, embodiments of the present invention can also include linear exogenous (donor) nucleic acid(s), compositions comprising these nucleic acids and methods of making and using these linear donor molecules. In certain embodiments, the linear donor molecule stably persists in the cell into which it is introduced. In other embodiments, the linear donor molecule is modified to resist exonucleolytic cleavage, for example by placing one or more phosphorothioate phosphodiester bonds between one or more base pairs on the ends of the donor molecule. The linear exogenous nucleic acid may also include single stranded specific DNA.

### NANOS2 Gene Editing

NANOS is an evolutionarily conserved family of RNA-binding proteins that are expressed specifically within the germ cells of both invertebrate and vertebrate animals. Ablation of the NANOS2 gene and its orthologs results in the loss of germ cells in Drosophila, C.elegans, Zebra fish, Xenopus, and mouse. In humans, germ cell loss and infertility are associated with mutations in a NANOS2 gene.

In vertebrates, three NANOS genes have been identified, amongst which NANOS2 and NANOS3 are expressed in PGCs. In mice, NANOS3 protein is first detectable in early PGCs, persists throughout their migration to the genital ridge, and then ceases by embryonic day 15.5 in males or prior to E13.5 in female embryos. In contrast, expression of NANOS2 is restricted to the male gonad. NANOS2 mRNA is first detectable in germ cells that have colonized the male embryonic gonad at around E13.0 after the germ cells begin to interact with gonadal somatic cells. Although the expression transiently decreases at later stages of embryogenesis, NANOS2 mRNA is detectable again in gonocytes during neonatal development.

Ablation of NANOS3 in mice results in the complete loss of germ cells for both sexes due to apoptotic cell death around E8.0. Importantly, inactivation of NANOS2 in mice results in loss of germ cells in male embryos only around E15.5. Thus, the germline is completely lacking at birth in male mice, but testicular somatic support cell populations are functionally intact. Also, NANOS2 null males and females are viable and grow to normal maturity. Moreover, NANOS2 null females are of normal fertility. Applicants have previously demonstrated that NANOS2 is expressed specifically by prospermatogonia (aka gonocytes) in pig embryos.

The NANOS family of genes is known and nucleotide sequences encoding the same are available through the NCBI Gene database or other such sources. Bovine NANOS genes are available at NM_0001281904.1 (NANOS2) (SEQ ID NO:2), NP 001268833.1 (NANOS2 amino acid sequence) (SEQ ID NO:1), XM_0052225796.5 (NANOS1) (SEQ ID NO:4), XP_005225853 (NANOS1 amino acid sequence) (SEQ ID NO: 3), and XM_05988542.1 (NANOS3) (SEQ ID NO:6) and XP_059744525.1 (NANOS3 amino acid sequence) (SEQ ID NO:5).

The present disclosure provides a genetically edited bovine animal or bovine animal cell comprising at least one edited chromosomal sequence encoding a NANOS2 protein or other protein associated with germ cell function or development. The edited chromosomal sequence may be (1) inactivated, (2) modified, or (3) comprise an integrated sequence. An inactivated chromosomal sequence is altered such that a NANOS2 protein function as it is related to prospermatogonial and spermatogonial cell development is impaired, reduced or eliminated. Thus, a genetically edited bovine animal comprising an inactivated chromosomal sequence may be termed a “knock-out” or a “conditional knock-out.” Similarly, a genetically edited bovine animal comprising an integrated sequence may be termed a “knock-in” or a “conditional knock-in.” Furthermore, a genetically edited bovine animal comprising a modified chromosomal sequence can comprise a targeted point mutation(s) or other modification such that an altered protein product is produced. Briefly, the process comprises introducing into an embryo or cell at least one RNA molecule encoding, for example, a targeted zinc finger nuclease or CRISPR/Cas9 complex and, optionally, at least one accessory polynucleotide. The method further comprises incubating the embryo or cell to allow expression of the zinc finger or Cas9 nuclease, wherein a double-stranded break introduced into the targeted chromosomal sequence by the zinc finger or Cas9 nuclease is repaired by an error-prone non-homologous end-joining DNA repair process or a homology-directed DNA repair process. The method of editing chromosomal sequences encoding a protein associated with germline development using targeted zinc finger nuclease or DRISPR/Cas9 technology is rapid, precise, and highly efficient.

In some embodiments of the present disclosure, at least one NANOS gene locus (e.g., a NANOS2 locus) is used as a target site for site specific editing. This can include insertion of an exogenous nucleic acid (e.g., a nucleic acid comprising a nucleotide sequence encoding a polypeptide of interest) or deletions of nucleic acids from the locus. In particular embodiments, an insertion and/or deletion modified locus is targeted. For example, integration of the exogenous nucleic acid and/or deletion of part of the genomic nucleic acid can modify the locus so as to produce a disrupted (i.e., inactivated) NANOS2 gene.

In some embodiments the edited NANOS2 locus can comprise a nucleotide sequence selected from the group of SEQ ID NOS: 50, 51, 52, 56, 57, 58, 59, 60, 61, 62, 63, 64, 65, 66, 67, 68, 69, 70, 71, 72, 73, 74, 75, 76, 77, 91, 92, 93, 94, 95, 96, 97, and 98. In some embodiments, an edited NANOS2 locus may comprise a nucleotide sequence that is substantially identical to a nucleotide sequence selected from the group consisting of SEQ ID NOs: 50, 51, 52, 56, 57, 58, 59, 60, 61, 62, 63, 64, 65, 66, 67, 68, 69, 70, 71, 72, 73, 74, 75, 76, 77, 91, 92, 93, 94, 95, 96, 97, and 98. For example, in some embodiments, a NANOS2 locus is a NANOS2 homologue (e.g., an ortholog or a paralog) that comprises a nucleotide sequence that is at least about 85% identical to a nucleotide sequence selected from the group consisting of SEQ ID NOs: 50, 51, 52, 56, 57, 58, 59, 60, 61, 62, 63, 64, 65, 66, 67, 68, 69, 70, 71, 72, 73, 74, 75, 76, 77, 91, 92, 93, 94, 95, 96, 97, and 98. A NANOS2 homologue may comprise a nucleotide sequence that is, for example and without limitation: at least 80%; at least 85%; at least about 90%; at least about 91%; at least about 92%; at least about 93%; at least about 94%; at least about 95%; at least about 96%; at least about 97%; at least about 98%; at least about 99%; at least about 99.5%; 99.6%, 99.7%, 99.8% and/or at least about 99.9% identical to about 20 contiguous nucleotides of a nucleotide sequence selected from the group consisting of SEQ ID NOs: 50, 51, 52, 56, 57, 58, 59, 60, 61, 62, 63, 64, 65, 66, 67, 68, 69, 70, 71, 72, 73, 74, 75, 76, 77, 91, 92, 93, 94, 95, 96, 97, and 98.

### Targeted Integration of a Nucleic Acid at a NANOS2 Locus

Site-specific integration of an exogenous nucleic acid at a NANOS2 locus can be accomplished by any technique known to those of skill in the art. In some embodiments, integration of an exogenous nucleic acid at a NANOS2 locus comprises contacting a cell (e.g., an isolated cell or a cell in a tissue or organism) with a nucleic acid molecule comprising the exogenous nucleic acid. In examples, such a nucleic acid molecule can comprise nucleotide sequences flanking the exogenous nucleic acid that facilitate homologous recombination between the nucleic acid molecule and at least one NANOS2 locus. In particular examples, the nucleotide sequences flanking the exogenous nucleic acid that facilitate homologous recombination may be complementary to endogenous nucleotides of the NANOS2 locus. In particular examples, the nucleotide sequences flanking the exogenous nucleic acid that facilitate homologous recombination can be complementary to previously integrated exogenous nucleotides. In some embodiments, a plurality of exogenous nucleic acids can be integrated at one NANOS2 locus, such as in gene stacking.

Integration of a nucleic acid at a NANOS2 locus can be facilitated (e.g., catalyzed) in some embodiments by endogenous cellular machinery of a host cell, such as, for example and without limitation, endogenous DNA and endogenous recombinase enzymes. In some embodiments, integration of a nucleic acid at a NANOS2 locus can be facilitated by one or more factors (e.g., polypeptides) that are provided to a host cell. For example, nuclease(s), recombinase(s), and/or ligase polypeptides may be provided (either independently or as part of a chimeric polypeptide) by contacting the polypeptides with the host cell, or by expressing the polypeptides within the host cell. Accordingly, in some examples, a nucleic acid comprising a nucleotide sequence encoding at least one nuclease, recombinase, and/or ligase polypeptide may be introduced into the host cell, either concurrently or sequentially with a nucleic acid to be integrated site-specifically at a NANOS2 locus, wherein the at least one nuclease, recombinase, and/or ligase polypeptide is expressed from the nucleotide sequence in the host cell.

### DNA-Binding Polypeptides

In some embodiments, site-specific integration can be accomplished by utilizing factors that are capable of recognizing and binding particular nucleotide sequences, for example, in the genome of a host organism. For instance, many proteins comprise polypeptide domains that are capable of recognizing and binding DNA in a site-specific manner. A DNA sequence that is recognized by a DNA-binding polypeptide can be referred to as a “target” sequence. Polypeptide domains that are capable of recognizing and binding DNA in a site-specific manner generally fold correctly and function independently to bind DNA in a site-specific manner, even when expressed in a polypeptide other than the protein from which the domain was originally isolated. Similarly, target sequences for recognition and binding by DNA-binding polypeptides are generally able to be recognized and bound by such polypeptides, even when present in large DNA structures (e.g., a chromosome), particularly when the site where the target sequence is located is one known to be accessible to soluble cellular proteins (e.g., a gene).

While DNA-binding polypeptides identified from proteins that exist in nature typically bind to a discrete nucleotide sequence or motif (e.g., a consensus recognition sequence), methods exist and are known in the art for modifying many such DNA-binding polypeptides to recognize a different nucleotide sequence or motif. DNA-binding polypeptides include, for example and without limitation: zinc finger DNA-binding domains; leucine zippers; UPA DNA-binding domains; GAL4; TAL; LexA; a Tet repressor; LacR; and a steroid hormone receptor.

In some examples, a DNA-binding polypeptide is a zinc finger. Individual zinc finger motifs can be designed to target and bind specifically to any of a large range of DNA sites. Canonical Cys₂His₂ (as well as non-canonical Cys₃His) zinc finger polypeptides bind DNA by inserting an alpha-helix into the major groove of the target DNA double helix. Recognition of DNA by a zinc finger is modular; each finger contacts primarily three consecutive base pairs in the target, and a few key residues in the polypeptide mediate recognition. By including multiple zinc finger DNA-binding domains in a targeting endonuclease, the DNA-binding specificity of the targeting endonuclease may be further increased (and hence the specificity of any gene regulatory effects conferred thereby may also be increased). See, e.g., Urnov et al. Nature 435:646-651 (2005). Thus, one or more zinc finger DNA-binding polypeptides can be engineered and utilized such that a targeting endonuclease introduced into a host cell interacts with a DNA sequence that is unique within the genome of the host cell.

Preferably, the zinc finger protein is non-naturally occurring in that it is engineered to bind to a target site of choice. See, for example, Beerli et al. Nature Biotechnol. 20:135-141 (2002); Pabo et al. Ann. Rev. Biochem. 70:313-340 (2001); Isalan et al. Nature Biotechnol. 19:656-660 (2001); Segal et al. Curr. Opin. Biotechnol. 12:632-637 (2001); Choo et al. Curr. Opin. Struct. Biol. 10:411-416 (2000); U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entirety.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 1998/37186; WO 1998/53057; WO 2000/27878; WO 2001/88197 and GB 2,338,237. In addition, enhancement of binding specificity for zinc finger binding domains has been described, for example, in co-owned WO 2002/077227.

In addition, as disclosed in these and other references, zinc finger domains and/or multi-fingered zinc finger proteins can be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein can include any combination of suitable linkers between the individual zinc fingers of the protein.

Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; WO 1995/19431; WO 1996/06166; WO 1998/53057; WO 1998/54311; WO 2000/27878; WO 2001/60970 WO 2001/88197; WO 2002/099084; WO 1998/53058; WO 1998/53059; WO 1998/53060; WO 2002/016536 and WO 2003/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein can include any combination of suitable linkers between the individual zinc fingers of the protein.

In some examples, a DNA-binding polypeptide is a DNA-binding domain from GAL4. GAL4 is a modular transactivator in Saccharomyces cerevisiae, but it also operates as a transactivator in many other organisms. See, e.g., Sadowski et al. Nature 335:563-564 (1988). In this regulatory system, the expression of genes encoding enzymes of the galactose metabolic pathway in S. cerevisiae is stringently regulated by the available carbon source. (Johnston Microbiol. Rev. 51:458-476 (1987)). Transcriptional control of these metabolic enzymes is mediated by the interaction between the positive regulatory protein, GAL4, and a 17-base pair symmetrical DNA sequence to which GAL4 specifically binds (the UAS).

Native GAL4 consists of 881 amino acid residues, with a molecular weight of 99 kDa. GAL4 comprises functionally autonomous domains, the combined activities of which account for activity of GAL4 in vivo. Ma and Ptashne Cell 48:847-853 (1987)); Brent and Ptashne Cell 43 (3 Pt 2):729-736 (1985). The N-terminal 65 amino acids of GAL4 comprise the GAL4 DNA-binding domain. Keegan et al. Science 231:699-704 (1986); Johnston Nature 328:353-355 (1987). Sequence-specific binding requires the presence of a divalent cation coordinated by 6 Cys residues present in the DNA binding domain. The coordinated cation-containing domain interacts with and recognizes a conserved CCG triplet at each end of the 17 bp UAS via direct contacts with the major groove of the DNA helix. Marmorstein et al. Nature 356:408-414 (1992). The DNA-binding function of the protein positions C-terminal transcriptional activating domains in the vicinity of the promoter, such that the activating domains can direct transcription.

Additional DNA-binding polypeptides that can be utilized in certain embodiments include, for example and without limitation, a binding sequence from a AVRBS3-inducible gene; a consensus binding sequence from a AVRBS3-inducible gene or synthetic binding sequence engineered therefrom (e.g., UPA DNA-binding domain); TAL; LexA (see, e.g., Brent and Ptashne (1985), supra); LacR (see, e.g., Labow et al. Mol. Cell. Biol. 10:3343-5336 (1990); Baim et al. Proc. Natl. Acad. Sci. USA 88(12):5072-5076 (1991)); a steroid hormone receptor (Ellliston et al. J. Biol. Chem. 265:11517-11521 (1990)); the Tet repressor (U.S. Pat. No. 6,271,341) and a mutated Tet repressor that binds to a tet operator sequence in the presence, but not the absence, of tetracycline (Tc); the DNA-binding domain of NF-.kappa.B; and components of the regulatory system described in Wang et al. Proc. Natl. Acad. Sci. USA 91(17):8180-8184 (1994), which utilizes a fusion of GAL4, a hormone receptor, and VP16.

In certain embodiments, the DNA-binding domain of one or more of the nucleases used in the methods and compositions described herein comprises a naturally occurring or engineered (non-naturally occurring) TAL effector DNA binding domain. See, e.g., U.S. Patent Publication No. 2011/0301073, incorporated by reference in its entirety herein.

In other embodiments, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., Mol. Microbiol. 43:1565-1575 (2002); Makarova et al., Nucl. Acids Res. 30:482-496 (2002); Makarova et al., Biol. Direct 1:7 (2006); Haft et al., PLoS Comput. Biol. 1:e60 (2005)) make up the gene sequences of the CRISPR/Cas nuclease system. CRISPR loci in microbial hosts contain a combination of Cas genes as well as non-coding RNA elements capable of programming the specificity of the CRISPR-mediated nucleic acid cleavage.

The Type II CRISPR is one of the most well characterized systems and carries out a targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Wastson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called “adaptation,” (ii) expression of the relevant proteins, as well as expression and processing of the array, followed by (iii) RNA-mediated interference with the foreign nucleic acid. Thus, in the bacterial cell, several Cas proteins are involved with the natural function of the CRISPR/Cas system and serve roles in functions such as insertion of a foreign DNA, and the like.

In certain embodiments, Cas protein can be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, can be obtainable from a cell or synthesized chemically or by a combination of these two procedures. The cell can be a cell that naturally produces a Cas protein, or a cell that naturally produces a Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce a Cas protein and is genetically engineered to produce the Cas protein.

In particular embodiments, a DNA-binding polypeptide specifically recognizes and binds to a target nucleotide sequence comprised within a genomic nucleic acid of a host organism. Any number of discrete instances of the target nucleotide sequence may be found in the host genome in some examples. The target nucleotide sequence may be rare within the genome of the organism (e.g., fewer than about 10, about 9, about 8, about 7, about 6, about 5, about 4, about 3, about 2, or about 1 copy(ies) of the target sequence may exist in the genome). For example, the target nucleotide sequence can be located at a unique site within the genome of the organism. Target nucleotide sequences can be, for example and without limitation, randomly dispersed throughout the genome with respect to one another; located in different linkage groups in the genome; located in the same linkage group; located on different chromosomes; located on the same chromosome; located in the genome at sites that are expressed under similar conditions in the organism (e.g., under the control of the same, or substantially functionally identical, regulatory factors); and located closely to one another in the genome (e.g., target sequences can be comprised within nucleic acids integrated as concatemers at genomic loci).

### Targeting Endonucleases

In particular embodiments, a DNA-binding polypeptide that specifically recognizes and binds to a target nucleotide sequence may be comprised within a chimeric polypeptide, so as to confer specific binding to the target sequence upon the chimeric polypeptide. In examples, such a chimeric polypeptide can comprise, for example and without limitation, nuclease, recombinase, and/or ligase polypeptides, as these polypeptides are described above. Chimeric polypeptides comprising a DNA-binding polypeptide and a nuclease, recombinase, and/or ligase polypeptide can also comprise other functional polypeptide motifs and/or domains, such as for example and without limitation: a spacer sequence positioned between the functional polypeptides in the chimeric protein; a leader peptide; a peptide that targets the fusion protein to an organelle (e.g., the nucleus); polypeptides that are cleaved by a cellular enzyme; peptide tags (e.g., Myc, His, and the like); and other amino acid sequences that do not interfere with the function of the chimeric polypeptide.

Functional polypeptides (e.g., DNA-binding polypeptides and nuclease polypeptides) in a chimeric polypeptide can be operatively linked. In some embodiments, functional polypeptides of a chimeric polypeptide can be operatively linked by their expression from a single polynucleotide encoding at least the functional polypeptides ligated to each other in-frame, so as to create a chimeric gene encoding a chimeric protein. In alternative embodiments, the functional polypeptides of a chimeric polypeptide can be operatively linked by other means, such as by cross-linkage of independently expressed polypeptides.

In some embodiments, a DNA-binding polypeptide, or guide RNA that specifically recognizes and binds to a target nucleotide sequence can be comprised within a natural isolated protein (or mutant thereof), wherein the natural isolated protein or mutant thereof also comprises a nuclease polypeptide (and can also comprise a recombinase and/or ligase polypeptide). Examples of such isolated proteins include TALENs, recombinases (e.g., Cre, Hin, Tre, and FLP recombinase), RNA-guided CRISPR/Cas9, and meganucleases.

As used herein, the term “targeting endonuclease” refers to natural or engineered isolated proteins and mutants thereof that comprise a DNA-binding polypeptide or guide RNA and a nuclease polypeptide, as well as to chimeric polypeptides comprising a DNA-binding polypeptide or guide RNA and a nuclease. Any targeting endonuclease comprising a DNA-binding polypeptide or guide RNA that specifically recognizes and binds to a target nucleotide sequence comprised within a NANOS2 locus (e.g., either because the target sequence is comprised within the native sequence at the locus, or because the target sequence has been introduced into the locus, for example, by recombination) can be utilized in certain embodiments.

Some examples of chimeric polypeptides that came be useful in particular embodiments of the invention include, without limitation, combinations of the following polypeptides: zinc finger DNA-binding polypeptides; a FokI nuclease polypeptide; TALE domains; leucine zippers; transcription factor DNA-binding motifs; and DNA recognition and/or cleavage domains isolated from, for example and without limitation, a TALEN, a recombinase (e.g., Cre, Hin, RecA, Tre, and FLP recombinases), RNA-guided CRISPR/Cas9, a meganuclease; and others known to those in the art. Particular examples include a chimeric protein comprising a site-specific DNA binding polypeptide and a nuclease polypeptide. Chimeric polypeptides can be engineered by methods known to those of skill in the art to alter the recognition sequence of a DNA-binding polypeptide comprised within the chimeric polypeptide, so as to target the chimeric polypeptide to a particular nucleotide sequence of interest.

In certain embodiments, the chimeric polypeptide comprises a DNA-binding domain (e.g., zinc finger, TAL-effector domain, etc.) and a nuclease (cleavage) domain. The cleavage domain can be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. Nucl. Acids Res. 25:3379-3388 (1997). Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5 to 8 nucleotides or by 15 to 18 nucleotides. However, any integral number of nucleotides, or nucleotide pairs, can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding, for example, such that one or more exogenous sequences (donors/trangsenes) are integrated at or near the binding (target) sites. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc. Natl. Acad. Sci. USA 89:4275-4279 (1992); Li et al. Proc. Natl. Acad. Sci. USA 90:2764-2768 (1993); Kim et al. Proc. Natl. Acad. Sci. USA 91:883-887 (1994); Kim et al. J. Biol. Chem. 269:31978-31982 (1994). Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. Proc. Natl. Acad. Sci. USA 95:10570-10575 (1998). Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in U.S. Patent Publication No. 2007/0134796, incorporated herein in its entirety. Additional restriction enzymes also contain separable binding and cleavage domains, and these are contemplated by the present disclosure. See, for example, Roberts et al. Nucl. Acids Res. 31:418-420 (2003).

In certain embodiments, the cleavage domain comprises one or more engineered cleavage half-domain (also referred to as dimerization domain mutants) that minimize or prevent homodimerization, as described, for example, in U.S. Patent Publication Nos. 2005/0064474; 2006/0188987 and 2008/0131962, the disclosures of all of which are incorporated by reference in their entirety herein.

Alternatively, nucleases can be assembled in vivo at the nucleic acid target site using so-called “split-enzyme” technology (see e.g., U.S. Patent Publication No. 2009/0068164). Components of such split enzymes may be expressed either on separate expression constructs or can be linked in one open reading frame where the individual components are separated, for example, by a self-cleaving 2A peptide or IRES sequence. Components can be individual zinc finger binding domains or domains of a meganuclease nucleic acid binding domain.

### Zinc Finger Nucleases

In specific embodiments, a chimeric polypeptide is a custom-designed zinc finger nuclease (ZFN) that may be designed to deliver a targeted site-specific double-strand DNA break into which an exogenous nucleic acid, or donor DNA, can be integrated (See US Patent publication 2010/0257638, incorporated by reference herein). ZFNs are chimeric polypeptides containing a non-specific cleavage domain from a restriction endonuclease (for example, FokI) and a zinc finger DNA-binding domain polypeptide. See, e.g., Huang et al. J. Protein Chem. 15:481-489 (1996); Kim et al. Proc. Natl. Acad. Sci. USA 94:3616-3620 (1997); Kim et al. Proc. Natl. Acad. Sci. USA 93:1156-1160 (1996); Kim et al. Proc. Natl. Acad. Sci. USA 91:883-887 (1994); Kim et al. Proc. Natl. Acad. Sci. USA 94:12875-12879 (1997); Kim et al. Gene 203:43-49 (1997); Kim et al. Biol. Chem. 379:489-495 (1998); Nahon and Raveh Nucl. Acids Res. 26:1233-1239 (1998); Smith et al. Nucl. Acids Res. 27:674-681 (1999). In some embodiments, the ZFNs comprise non-canonical zinc finger DNA binding domains (see US Patent publication 2008/0182332, incorporated by reference herein). The FokI restriction endonuclease must dimerize via the nuclease domain in order to cleave DNA and introduce a double-strand break. Consequently, ZFNs containing a nuclease domain from such an endonuclease also require dimerization of the nuclease domain in order to cleave target DNA. Mani et al. Biochem. Biophys. Res. Commun. 334:1191-1197 (2005); Smith et al. Nucl. Acids Res. 28:3361-3369 (2000). Dimerization of the ZFN can be facilitated by two adjacent, oppositely oriented DNA-binding sites. Id.

In particular examples, a method for the site-specific integration of an exogenous nucleic acid into at least one NANOS2 locus of a host comprises introducing into a host cell a ZFN, wherein the ZFN recognizes and binds to a target nucleotide sequence, wherein the target nucleotide sequence is comprised within at least one NANOS2 locus of the host. In certain examples, the target nucleotide sequence is not comprised within the genome of the host at any other position than the at least one NANOS2 locus. For example, a DNA-binding polypeptide of the ZFN can be engineered to recognize and bind to a target nucleotide sequence identified within the at least one NANOS2 locus (e.g., by sequencing the NANOS2 locus). A method for the site-specific integration of an exogenous nucleic acid into at least one NANOS2 performance locus of a host that comprises introducing into a cell of the host a ZFN can also comprise introducing into the cell an exogenous nucleic acid, wherein recombination of the exogenous nucleic acid into a nucleic acid of the host comprising the at least one NANOS2 locus is facilitated by site-specific recognition and binding of the ZFN to the target sequence (and subsequent cleavage of the nucleic acid comprising the NANOS locus).

### Optional Exogenous Nucleic Acids for Integration at a NANOS2 Locus

Embodiments of the disclosure can include one or more nucleic acids selected from the group consisting of: an exogenous nucleic acid for site-specific integration in at least one NANOS2 locus, for example and without limitation, an ORF; a nucleic acid comprising a nucleotide sequence encoding a targeting endonuclease; and a vector comprising at least one of either or both of the foregoing. Thus, particular nucleic acids for use in some embodiments include nucleotide sequences encoding a polypeptide, structural nucleotide sequences, and/or DNA-binding polypeptide recognition and binding sites.

### Optional Exogenous Nucleic Acid Molecules for Site-Specific Integration

As noted above, insertion of an exogenous sequence (also called a “donor sequence” or “donor” or “transgene”) is provided, for example for expression of a polypeptide, correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Additionally, a donor sequence can comprise a vector molecule containing sequences that are not homologous to the region of interest in cellular chromatin. A donor molecule can contain several, discontinuous regions of homology to cellular chromatin. For example, for targeted insertion of a sequence not normally present in a region of interest, said sequence can be present in a donor nucleic acid molecule and flanked by regions of homology to a sequence in the region of interest.

The donor polynucleotide can be DNA or RNA, single-stranded or double-stranded and can be introduced into a cell in linear or circular form. See e.g., U.S. Patent Appl. Publication Nos. 2010/0047805, 2011/0281361, 2011/0207221 and U.S. Pat. No. 10,174,331. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. Proc. Natl. Acad. Sci. USA 84:4959-4963 (1987); Nehls et al. Science 272:886-889 (1996). Additional methods for protecting exogenous polynucleotides from degradation include, but are not limited to, the addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally integrated so that its expression is driven by the endogenous promoter at the integration site, namely the promoter that drives expression of the endogenous gene into which the donor is integrated (e.g., NANOS2). However, it will be apparent that the donor may comprise a promoter and/or enhancer, for example a constitutive promoter or an inducible or tissue specific promoter.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

Exogenous nucleic acids that may be integrated in a site-specific manner into at least one NANOS2 locus, so as to modify the NANOS2 locus, in embodiments include, for example and without limitation, nucleic acids comprising a nucleotide sequence encoding a polypeptide of interest; nucleic acids comprising an agronomic gene; nucleic acids comprising a nucleotide sequence encoding an RNAi molecule; or nucleic acids that disrupt the NANOS2 gene.

In some embodiments, an exogenous nucleic acid is integrated at a NANOS2 locus, so as to modify the NANOS2 locus, wherein the nucleic acid comprises a nucleotide sequence encoding a polypeptide of interest, such that the nucleotide sequence is expressed in the host from the NANOS2 locus. In some examples, the polypeptide of interest (e.g., a foreign protein) is expressed from a nucleotide sequence encoding the polypeptide of interest in commercial quantities. In such examples, the polypeptide of interest may be extracted from the host cell, tissue, or biomass.

### Nucleic Acid Molecules Comprising a Nucleotide Sequence Encoding a Targeting Endonuclease

In some embodiments, a nucleotide sequence encoding a targeting endonuclease can be engineered by manipulation (e.g., ligation) of native nucleotide sequences encoding polypeptides comprised within the targeting endonuclease. For example, the nucleotide sequence of a gene encoding a protein comprising a DNA-binding polypeptide can be inspected to identify the nucleotide sequence of the gene that corresponds to the DNA-binding polypeptide, and that nucleotide sequence can be used as an element of a nucleotide sequence encoding a targeting endonuclease comprising the DNA-binding polypeptide. Alternatively, the amino acid sequence of a targeting endonuclease can be used to deduce a nucleotide sequence encoding the targeting endonuclease, for example, according to the degeneracy of the genetic code.

In exemplary nucleic acid molecules comprising a nucleotide sequence encoding a targeting endonuclease, the last codon of a first polynucleotide sequence encoding a nuclease polypeptide, and the first codon of a second polynucleotide sequence encoding a DNA-binding polypeptide, can be separated by any number of nucleotide triplets, e.g., without coding for an intron or a “STOP.” Likewise, the last codon of a nucleotide sequence encoding a first polynucleotide sequence encoding a DNA-binding polypeptide, and the first codon of a second polynucleotide sequence encoding a nuclease polypeptide, may be separated by any number of nucleotide triplets. In these and further embodiments, the last codon of the last (i.e., most 3′ in the nucleic acid sequence) of a first polynucleotide sequence encoding a nuclease polypeptide, and a second polynucleotide sequence encoding a DNA-binding polypeptide, may be fused in phase-register with the first codon of a further polynucleotide coding sequence directly contiguous thereto, or separated therefrom by no more than a short peptide sequence, such as that encoded by a synthetic nucleotide linker (e.g., a nucleotide linker that may have been used to achieve the fusion). Examples of such further polynucleotide sequences include, for example and without limitation, tags, targeting peptides, and enzymatic cleavage sites. Likewise, the first codon of the most 5′ (in the nucleic acid sequence) of the first and second polynucleotide sequences may be fused in phase-register with the last codon of a further polynucleotide coding sequence directly contiguous thereto or separated therefrom by no more than a short peptide sequence.

A sequence separating polynucleotide sequences encoding functional polypeptides in a targeting endonuclease (e.g., a DNA-binding polypeptide and a nuclease polypeptide) can, for example, consist of any sequence, such that the amino acid sequence encoded is not likely to significantly alter the translation of the targeting endonuclease. Due to the autonomous nature of known nuclease polypeptides and known DNA-binding polypeptides, intervening sequences will not, for example, interfere with the respective functions of these structures.

### CRISPR/Cas9 Based Gene Editing of Bovine Zygotes by Electroporation

Despite the relative simplicity of techniques for designing and producing CRISPR/Cas9 reagents for editing, the delivery of these into mammalian zygote stage embryos or other cells can be challenging. The most common approach for introducing foreign nucleic acids into mammalian embryos is microinjection which requires a level of technical expertise that is often difficult to master, especially with livestock animals.

To overcome the difficulties of microinjection, electroporation-based protocols have been devised for the introduction of CRISPR/Cas9 reagents into mammals. The following method comprises electroporation into zygotes of preassembled sgRNA/Cas9 ribonucleoproteins (RNPs) followed by embryo culture to the blastocyst stage and then non-surgical embryo transfer to efficiently generate mutant animals. (Miao et al., Biol. Repro. 101(1):177-187 (2019); Ciccarelli et al., Proc. Natl. Acad. Sci. USA 117(39):24195-24204 (2020); both incorporated herein by reference in their entirety).

Dual sgRNAs are designed to delete a large portion of the coding sequence (CDS) for the bovine NANOS2 gene. Candidate sgRNAs are designed and off-target predictor scores ranked using a software program (e.g., CRISPOR or CRISPR Design). Sense and antisense sgRNAs are designed to target approximately 20 nucleotides upstream of the PAM sequences in the sense and antisense strand and adjacent to the start or stop codon. All selected sgRNAs should have an off-target predictor score of >80.

For the generation of RNP complexes, sgRNAs (100 ng/μL each) and Cas9 protein (200 ng/μL) are diluted in buffer (TE buffer) at a 1:1 mass ratio. Medium is added (e.g., Opti-MEM Reduced Serum media with no phenol red) to provide a final reaction volume of about 20 μL (containing 4000 ng of sgRNA and 4000 ng of Cas9 protein), and the mixture can be incubated at room temperature for about 10 min before being used as the electroporation solution.

Bovine cumulus-oocyte complexes (COCs) at about 0.5 days post conception are recovered in medium (e.g., DMEM/F-12 HEPES medium) with fetal bovine serum from dissected ampulla or aspiraction of ovarian follicles. In vitro matured COCs are washed and placed in equilibrated in vitro fertilization medium (e.g., BO-IVF; IVF Biosciences) under embryo-safe mineral oil. Cryopreserved or fresh bovine semen are thawed and viable sperm subsequently isolated using centrifugal gradient separation (e.g., a 1 mL column of 80% BoviPure®) and centrifuged at 500×g for 15 min. The resulting sperm pellet is washed by centrifugation and resuspended in IVF medium. At 20 to 24 hours post onset of maturation, 50 μL of sperm suspension is added to IVF medium containing about 50 matured oocytes and incubated at 38.5° C. in an atmosphere of 5% CO₂ in air for approximately 18 hours. Presumptive zygotes are denuded by mixing in PBS (e.g., Dulbecco's PBS) containing 0.1% hyaluronidase before being subjected to electroporation.

At 18 to 22 hours post IVF, zygotes (approximately 10 to 100) are washed with medium (e.g., Opti-MEM) and transferred to an electroporation vessel, such as a cuvette, containing electroporation solution (sgRNA-Cas9 RNP complexes in medium). Electroporation can be carried out using an electroporation system (e.g., BTX T820 Square Wave Electroporation system). The typical conditions are 2 pulses of 20 V/3 ms pulse length (Miao et al., Biol. Reprod. 101(1):177-187, 1019). Following electroporation, embryos are recovered from the electroporation vessel by washing (TL-HEPES), equilibrated, and cultured to the blastocyst stage (equilibrated BO-IVC medium under embryo-safe mineral oil at 38.5° C. in an atmosphere of 5% CO₂ and 5% O₂).

The CRISPR/Cas9 edited embryos can be transferred to pseudopregnant recipient cows for pregnancy establishment. Genotyping of offspring is carried out by PCR of genomic DNA from, for example, blood cells or hair follicle cells. A reduced size of amplicons in the NANOS2 CRISPR/Cas9 animals demonstrate the presence of deletion mutations in the genes.

### Other Knock-Out Methods

Various other techniques known in the art can be used to inactivate genes to make knock-out animals and/or to introduce nucleic acid constructs into animals to produce founder animals and to make animal lines, in which the knock-out or nucleic acid construct is integrated into the genome. Such techniques include, without limitation, pronuclear microinjection (U.S. Pat. No. 4,873,191), retrovirus mediated gene transfer into germ lines (Van der Putten et al., Proc. Natl. Acad. Sci. USA 82:6148-1652 (1985)), gene targeting into embryonic stem cells (Thompson et al., Cell 56; 313-321 (1989)), electroporation of embryos (Lo, Mol. Cell. Biol. 3:1803-1814 (1983)), sperm-mediated gene transfer (Lavitrano et al., Proc. Natl. Acad. Sci. USA 99:14230-14235 (2002); Lavitrano et al., Reprod. Fert. Develop. 18:19-23 (2006)), and in vitro transformation of somatic cells, such as cumulus or mammary cells, or adult, fetal, or embryonic stem cells, followed by nuclear transplantation (Wilmut et al. Nature 385:810-813 (1997); and Wakayama et al., Nature 394:369-374 (1998)). Pronuclear microinjection, sperm mediated gene transfer, and somatic cell nuclear transfer are particularly useful techniques. An animal that is genomically modified is an animal wherein all its cells have the genetic modification, including its germ line cells. When methods are used that produce a bovine animal that is mosaic in its genetic modification, the animals can be inbred and progeny that are genomically modified can be selected. Cloning, for instance, can be used to make a mosaic bovine animal if its cells are modified at the blastocyst state, or genomic modification can take place when a single-cell is modified. Bovine animals that are modified so they do not sexually mature can be homozygous or heterozygous for the modification, depending on the specific approach that is used. If a particular gene was inactivated by a knock-out modification, homozygousity would normally be required. If a particular gene was inactivated by an RNA interference or dominant negative strategy, then heterozygosity is often adequate.

Typically, in embryo/zygote microinjection, a nucleic acid construct or mRNA is introduced into a fertilized egg; 1- or 2-cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of a donor porcine or bovine animal). In vitro fertilized eggs can be produced as follows. For example, swine and/or bovine ovaries can be collected at an abattoir and maintained at 22 to 28° C. during transport. Ovaries can be washed and isolated for follicular aspiration, and follicles ranging from 4 to 8 mm can be aspirated into 50 mL conical centrifuge tubes using an 18-gauge needle and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 Oocyte Maturation Medium (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/mL cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

Linearized nucleic acid constructs or mRNA can be injected into one of the pronuclei or into the cytoplasm. Then the injected eggs can be transferred to a recipient female (e.g., into the oviducts of a recipient female) and allowed to develop in the recipient female to produce the genetically edited animals. In particular, in vitro fertilized embryos can be centrifuged at 15,000×g for 5 minutes to sediment lipids allowing visualization of the pronucleus. The embryos can be injected using, for example, an Eppendorf™ FemtoJet® injector and can be cultured until blastocyst formation. Rates of embryo cleavage and blastocyst formation and quality can be recorded.

Embryos can be surgically transferred into uteri of asynchronous recipients. Typically, 100 to 200 (e.g., 150 to 200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic cell (e.g., a transgenic bovine cell or porcine cell) such as an embryonic blastomere, fetal fibroblast, adult ear fibroblast, or granulosa cell that includes a nucleic acid construct described above, can be introduced into an enucleated oocyte to establish a combined cell. Oocytes can be enucleated by partial zona dissection near the polar body and then pressing out cytoplasm at the dissection area. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al., Science 280:1256-1258 (1998) and U.S. Pat. No. 6,548,741. For pigs, recipient females can be checked for pregnancy approximately 20 to 21 days after transfer of the embryos.

Standard breeding techniques can be used to create bovine animals or pigs that are homozygous for the exogenous nucleic acid from the initial heterozygous founder animals. Homozygosity may not be required, however. Genetically edited bovines described herein can be bred with other bovines of interest.

In some embodiments, a nucleic acid of interest and a selectable marker can be provided on separate transposons and provided to either embryos or cells in unequal amount, where the amount of transposon containing the selectable marker far exceeds (5- to 10-fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic or genetically edited animals can be produced that are not constrained to retain selectable markers in subsequent generations, an issue of some concern from a public safety perspective.

Once a gene edited animal has been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., 1989, Molecular Cloning, A Laboratory Manual, second edition, Cold Spring Harbor Press, Plainview; N.Y. Polymerase chain reaction (PCR) techniques also can be used in the initial screening PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA, including sequences from total genomic DNA or total cellular RNA. Primers typically are 14 to 40 nucleotides in length but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis Genetic Engineering News 12:1 (1992); Guatelli et al. Proc. Natl. Acad. Sci. USA 87:1874 (1990); and Weiss Science 254:1292 (1991). At the blastocyst stage, embryos can be individually processed for analysis by PCR, Southern hybridization and splinkerette PCR (see, e.g., Dupuy et al., Proc Natl Acad Sci USA 99:4495 (2002).

Expression of a nucleic acid sequence encoding a polypeptide in the tissues of transgenic or genetically edited bovine and/or porcine animals can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) systems are known. Double-stranded RNA (dsRNA) induces sequence-specific degradation of homologous gene transcripts. RNA-induced silencing complex (RISC) metabolizes dsRNA to small 21- to 23-nucleotide small interfering RNAs (siRNAs). RISC contains a double stranded RNAse (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of inactivating a gene in a genetically edited animal comprises inducing RNA interference against a target gene and/or nucleic acid such that expression of the target gene and/or nucleic acid is reduced.

For example, the exogenous nucleic acid sequence can induce RNA interference against a nucleic acid encoding a polypeptide. For example, double-stranded small interfering RNA (siRNA) or small hairpin RNA (shRNA) homologous to a target DNA can be used to reduce expression of that DNA. A construct for siRNA can be produced as described, for example, in Fire et al. Nature 391:806 (1998); Romano and Masino Mol. Microbiol. 6:3343 (1992); Cogoni et al. EMBO J. 15:3153 (1996); Cogoni and Masino Nature 399:166 (1999); Misquitta and Paterson Proc. Natl. Acad. Sci. USA 96:1451 (1999); and Kennerdell and Carthew Cell 95:1017 (1998). Constructs for shRNA can be produced as described by McIntyre and Fanning BMC Biotechnology 6:1 (2006). In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically edited animal such as a livestock animal, and in particular, pigs and bovine animals, that express an RNAi directed against a neuroendocrine gene selective for sexual maturation. An embodiment is an RNAi directed against a gene in the group consisting of Gpr54, Kiss1, and GnRH1. The RNAi can be, for instance, a siRNA, a shRNA, a dsRNA, a RISC or a miRNA.

### Inducible Systems

An inducible system can be used to control the expression of a sexual maturation gene. Various inducible systems are known that allow spatiotemporal control of expression of a gene. Several have been proven to be functional in vivo in gene edited animals.

An example of an inducible system is the tetracycline (tet)-on promoter system, which can be used to regulate transcription of a polynucleic acid. In this system, a mutated Tet repressor (TetR) is fused to the activation domain of herpes simplex virus VP 16 trans-activator protein to create a tetracycline-controlled transcriptional activator (tTA), which is regulated by tet or doxycycline (dox). In the absence of antibiotic transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically edited animals. One animal line expresses the activator (tTA, rtTA, or Cre recombinase) under the control of a selected promoter. Another set of genetically edited animals express the acceptor, in which the expression of the gene of interest (or the gene to be modified) is under the control of the target sequence for the tTA/rtTA transactivators (or is flanked by loxP sequences). Mating the two strains of animals provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed foxed DNA) is excised by Cre-mediated recombination. Control of Cre expression in a transgenic animal, using either spatial control (with a tissue- or cell-specific promoter), or temporal control (with an inducible system), results in control of DNA excision between the two loxP sites. One application is for conditional gene inactivation (conditional knock-out). Another approach is for protein over-expression, wherein a floxed stop codon is inserted between the promoter sequence and the DNA of interest. Genetically edited animals do not express the transgene until Cre is expressed, leading to excision of the floxed stop codon. This system has been applied to tissue-specific oncogenesis and controlled antigene receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically edited animal such as a pig or bovine animal that comprise a neuroendocrine gene selective for sexual maturation that is under control of an inducible system. The genetic modification of an animal can be genomic or mosaic. An embodiment is a gene in the group consisting of Gpr54, Kiss1, and GnRH1 that is under control of an inducible system. The inducible system can be, for instance, selected from the group consisting of Tet-On, Tet-Off, Cre-lox, and Hif1 alpha.

### Vectors and Nucleic Acids

A variety of nucleic acids can be introduced into cells for knock-out purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller Antisense Nucleic Acid Drug Dev. 7(3):187 (1997); and Hyrup et al., Bioorgan. Med. Chem. 4:5 (1996). In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, inducible promoters, and promoters responsive or unresponsive to a particular stimulus. Suitable tissue specific promoters can result in preferential expression of a nucleic acid transcript in beta cells and include, for example, the human insulin promoter. Other tissue specific promoters can result in preferential expression in, for example, hepatocytes or heart tissue and can include the albumin or alpha-myosin heavy chain promoters, respectively. In other embodiments, a promoter that facilitates the expression of a nucleic acid molecule without significant tissue or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al., Hum. Gene Ther. 12:563 (2001); and Kiwaki et al., Hum. Gene Ther. 7:821 (1996).

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they can increase expression by affecting transcription, stability of the mRNA, translational efficiency, and the like. Such regulatory regions can be included in a nucleic acid construct as desired to obtain optimal expression of the nucleic acids in the cell(s). Sufficient expression, however, can sometimes be obtained without such additional elements.

A nucleic acid construct can be used that encodes a signal peptide or a selectable marker. A signal peptide can be used such that an encoded polypeptide is directed to a particular cellular location (e.g., the cell surface). Non-limiting examples of a selectable marker include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein (GFP) or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban et al., Proc. Natl. Acad. Sci. 89:6861 (1992), for a review of Cre/lox technology, and Brand and Dymecki Dev. Cell 6:7 (2004). A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in F0 animals (e.g., bovine animals, pigs, other livestock animals). Tissue specific activation of the transgene can be accomplished, for example, by crossing a bovine animal or pig that ubiquitously expresses a marker-interrupted transgene to a bovine animal or pig, respectively, expressing Cre or Flp in a tissue-specific manner, or by crossing a bovine animal or pig that expresses a marker-interrupted transgene in a tissue-specific manner to a bovine animal or pig, respectively, that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST), a His tag, and a FLAG™ tag (Kodak, New Haven, Conn.).

Nucleic acid constructs can be methylated using an SssI CpG methylase (New England Biolabs, Ipswich, Mass.). In general, the nucleic acid construct can be incubated with S-adenosylmethionine and SssI CpG-methylase in buffer at 37° C. Hypermethylation can be confirmed by incubating the construct with one unit of HinP1I endonuclease for 1 hour at 37° C. and assaying by agarose gel electrophoresis.

Nucleic acid constructs can be introduced into embryonic, fetal, or adult animal cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In a transposon system, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al., Nucl. Acids Res. 31:6873 (2003)); Tol2 (Kawakami Genome Biol. 8(Suppl.1):S7 (2007); Minos (Pavlopoulos et al., Genome Biol. 8(Suppl.1):S2 (2007)); Hsmar1 (Miskey et al. Mol. Cell Biol. 27:4589 (2207)); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Insulator elements also can be included in a nucleic acid construct to maintain expression of the exogenous nucleic acid and to inhibit the unwanted transcription of host genes. See, for example, U.S. Publication No. 2004/0203158. Typically, an insulator element flanks each side of the transcriptional unit and is internal to the inverted repeat of the transposon. Non-limiting examples of insulator elements include the matrix attachment region-(MAR) type insulator elements and border-type insulator elements. See, for example, U.S. Pat. Nos. 6,395,549, 5,731,178, 6,100,448, and 5,610,053, and U.S. Patent Appl. Publication No. 2004/0203158.

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically edited organism or genetically engineered organism whose genetic material has been altered using genetic engineering techniques. A knock-out bovine animal or pig is thus transgenic or genetically edited regardless of whether or not exogenous genes or nucleic acids are expressed in the animal or its progeny.

### Founder Animals, Animal Lines, Traits, and Reproduction

Founder bovine animals and/or pigs can be produced by cloning and other methods described herein. The founders can be homozygous for a genetic modification, as in the case where a zygote or a primary cell undergoes a homozygous modification. Similarly, founders can also be made that are heterozygous. In the case of NANO2 knock-outs, the founders are preferably heterozygous. In the case of NANOS2 knock-outs, the founders are preferably heterozygous. The founders can be genomically modified, meaning that all the cells in their genome have undergone modification. Founders can be mosaic for a modification, as can happen when vectors are introduced into one of a plurality of cells in an embryo, typically at a blastocyst stage. Founders can be mosaic for a modification, as can happen when vectors are introduced into one of a plurality of cells in an embryo, typically, at a blastocyst stage. Progeny of mosaic bovine animals and/or pigs can be tested to identify progeny that are genomically modified. A bovine animal and/or pig line is established when a pool of animals has been created that can be reproduced sexually or by assisted reproductive techniques, with heterogeneous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Sci. 40:123-137 (1994); U.S. Pat. No. 7,709,206, US Patent Apl. publication 2001/0016315, US 2011/0023140, and US 2005/0153317. An animal line can include a trait chosen from a trait in the group consisting of a production trait, a type-trait, a workability trait, a fertility trait, a mothering trait, and a disease resistance trait. Further traits include expression of a recombinant gene product.

Animals with a desired trait or traits can be modified to prevent their sexual maturation. Since the animals are sterile until matured, it is possible to regulate sexual maturity as a means of controlling the dissemination of the animals. Animals that have been bred or modified to have one or more traits can thus be provided to recipients with a reduced risk that the recipients will breed the animals and appropriate the value of the traits to themselves. Embodiments of the disclosure include genetically modifying a genome of an animal with the modification comprising an inactivated sexual maturation gene, wherein the sexual maturation gene in a wild-type animal expresses a factor selective for sexual maturation. Embodiments include treating the animal by administering a compound to remedy a deficiency caused by the loss of expression of the gene to induce sexual maturation in the animal.

Breeding of animals that require administration of a compound to induce sexual maturity may advantageously be accomplished at a treatment facility. The treatment facility can implement standardized protocols on well-controlled stock to efficiently produce consistent animals. Animal progeny can be distributed to a plurality of locations to be raised. Farms and farmers (a term including a ranch and ranchers) can thus order a desired number of progeny with a specified range of ages and/or weights and/or traits and have them delivered at a desired time and/or location. The recipients, e.g., farmers, can then raise the animals and deliver them to market as they desire.

Embodiments include delivering (e.g., to one or more locations, to a plurality of farms) a genetically edited livestock animal having an inactivated neuroendocrine gene selective for sexual maturation. Embodiments include delivery of animals having an age of between about 1 day and about 180 days. The animal can have one or more traits (for example one that expresses a desired trait or a high-value trait or a novel trait or a recombinant trait). Embodiments further include providing said animal and/or breeding said animal.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention. The following examples are provided to illustrate certain particular features and/or embodiments. The examples should not be construed to limit the disclosure to the particular features or embodiments exemplified.

### Example 1

### Design, Construction and Testing of Bovine NANOS2 CRISPR Reagents for Embryonic Fibroblasts

Potential target sites for sgRNAs were initially identified based on the presence of protospacer adjacent motif (PAM) sequences within either the coding sequence of the bovine NANOS2 gene or the sequence immediately flanking the coding sequence. Each potential site was analysed for potential off-target binding by using the BLAST algorithm to analyse the bovine genome for sequence matches. Nine potential sgRNA-binding sites were selected (three 5′ to the coding sequence, three within the coding sequence, and three 3′ to the stop codon) that appeared to have high specificity for the NANOS2 gene within the bovine genome.

For each identified sgRNA binding site, 2 guide sequences were designed; a 20-mer binding sequence, and a 19-, 18- or 17-mer binding sequence. See Table 1 and FIG. 5

TABLE 1

| | | |
|---|---|---|
| oSL48 | caccggtctttgggaatataaaag | forward oligo for bovine NANOS2 5′guide 1 20-mer |
| oSL49 | aaacatttatattcccaaagacc | reverse oligo for bovine NANOS2 5′guide 1 20-mer |
| oSL50 | caccgtctttgggaatataaaag | forward oligo for bovine NANOS2 5′guide 1 19-mer |
| oSL51 | aaacatttatattcccaaagac | reverse oligo for bovine NANOS2 5′guide 1 19-mer |
| oSL52 | caccgattgcttagaagggtcttt | forward oligo for bovine NANOS2 5′guide 2 20-mer |
| oSL53 | aaacaaagacccttctaagcaaagc | reverse oligo for bovine NANOS2 5′guide 2 20-mer |
| oSL54 | caccgttgcttagaagggtcttt | forward oligo for bovine NANOS2 5′guide 2 18-mer |
| oSL55 | aaacaaagacccttctaagcaac | reverse oligo for bovine NANOS2 5′guide 2 18-mer |
| oSL56 | caccgagctccacctttgcttagaa | forward oligo for bovine NANOS2 5′guide 3 20-mer |
| oSL57 | aaacttctaagcaaaggtggagctc | reverse oligo for bovine NANOS2 5′guide 3 20-mer |
| oSL58 | caccgctccacctttgcttagaa | forward oligo for bovine NANOS2 5′guide 3 19-mer |
| oSL59 | aaacttctaagcaaaggtggagc | reverse oligo for bovine NANOS2 5′guide 3 19-mer |
| oSL60 | caccggcaagggttggagacccaa | forward oligo for bovine NANOS2 internal guide 1 20-mer |
| oSL61 | aaacttgggtaccaacccttgcc | reverse oligo for bovine NANOS2 internal guide 1 20-mer |
| oSL62 | caccgcaagggttggagacccaa | forward oligo for bovine NANOS2 internal guide 1 19-mer |
| oSL63 | aaacttgggtaccaacccttgc | reverse oligo for bovine NANOS2 internal guide 1 19-mer |
| oSL64 | caccgccgagacccgggccccatg | forward oligo for bovine NANOS2 internal guide 2 20-mer |
| oSL65 | aaaccatggggcccgggtctcggc | reverse oligo for bovine NANOS2 internal guide 2 20-mer |
| oSL66 | caccgagacccgggccccatg | forward oligo for bovine NANOS2 internal guide 2 17-mer |
| oSL67 | aaaccatggggcccgggtctc | reverse oligo for bovine NANOS2 internal guide 2 17-mer |
| oSL68 | caccgcccatgtggagcaggatcag | forward oligo for bovine NANOS2 internal guide 3 20-mer |
| oSL69 | aaacctgatcctgctccacatgggc | reverse oligo for bovine NANOS2 internal guide 3 20-mer |
| oSL70 | caccgcatgtggagcaggatcag | forward oligo for bovine NANOS2 internal guide 3 18-mer |
| oSL71 | aaacctgatcctgctccacatgc | reverse oligo for bovine NANOS2 internal guide 3 18-mer |
| oSL72 | caccgtccccatctctggactcgtc | forward oligo for bovine NANOS2 3′guide 1 20-mer |
| oSL73 | aaacgacgagtccagagatggggac | reverse oligo for bovine NANOS2 3′guide 1 20-mer |
| oSL74 | caccgcccatctctggactcgtc | forward oligo for bovine NANOS2 3′guide 1 18-mer |
| oSL75 | aaacgacgagtccagagatgggc | reverse oligo for bovine NANOS2 3′guide 1 18-mer |
| oSL76 | caccgtctggactcgtccggccctt | forward oligo for bovine NANOS2 3′guide 2 20-mer |
| oSL77 | aaacaagggccggacgagtccagac | reverse oligo for bovine NANOS2 3′guide 2 20-mer |
| oSL78 | caccggactcgtccggccctt | forward oligo for bovine NANOS2 3′guide 2 17-mer |
| oSL79 | aaacaagggccggacgagtcc | reverse oligo for bovine NANOS2 3′guide 2 17-mer |
| oSL80 | caccgtacagtacttgatccctgc | forward oligo for bovine NANOS2 3′guide 3 20-mer |
| oSL81 | aaacgcagggatcaagtactgagac | reverse oligo for bovine NANOS2 3′guide 3 20-mer |
| oSL82 | caccgtcagtacttgatccctgc | forward oligo for bovine NANOS2 3′guide 3 18-mer |
| oSL83 | aaacgcagggatcaagtactgac | reverse oligo for bovine NANOS2 3′guide 3 18-mer |

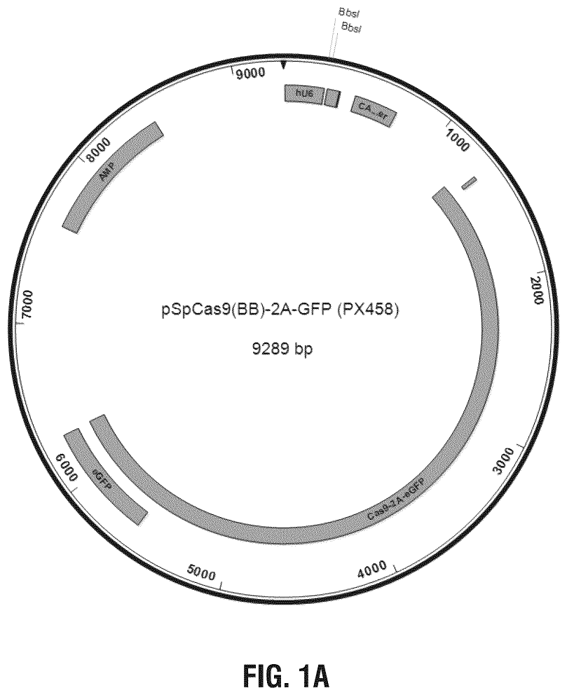

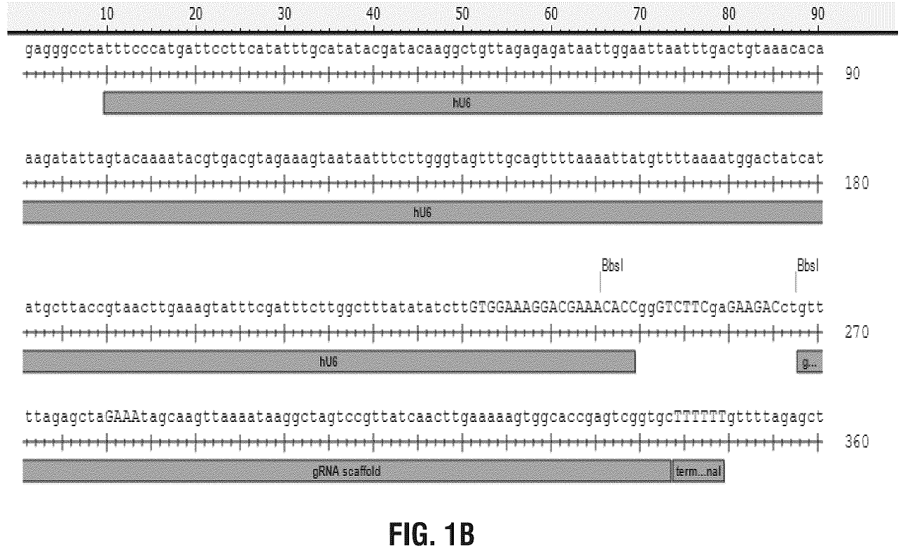

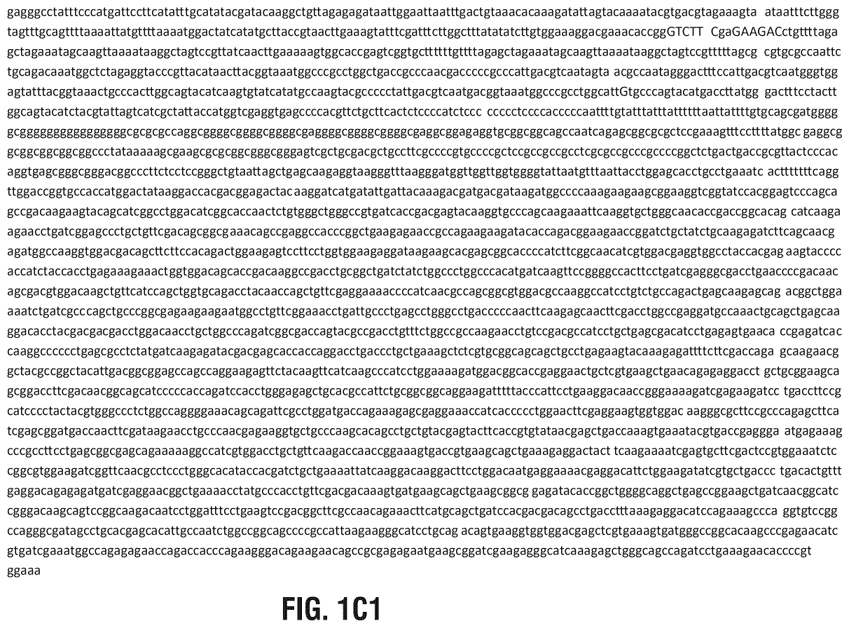

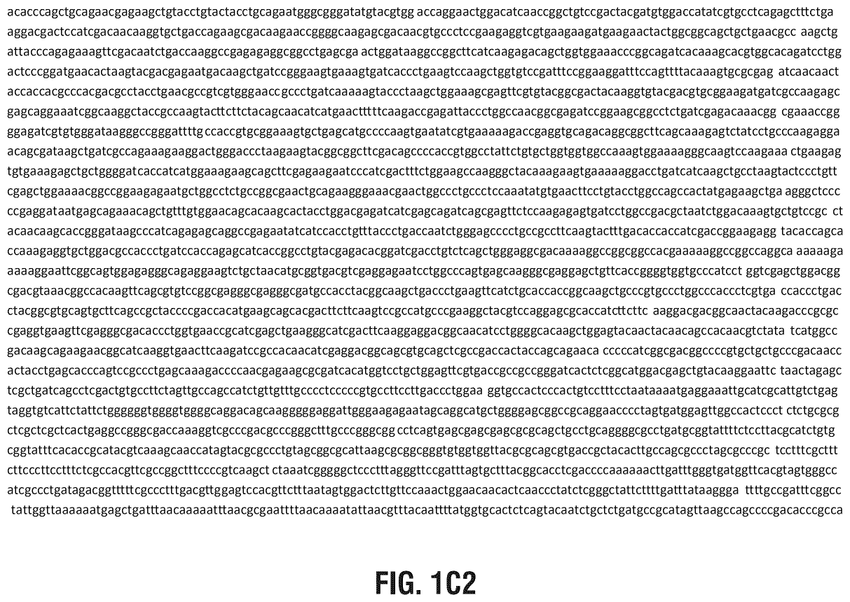

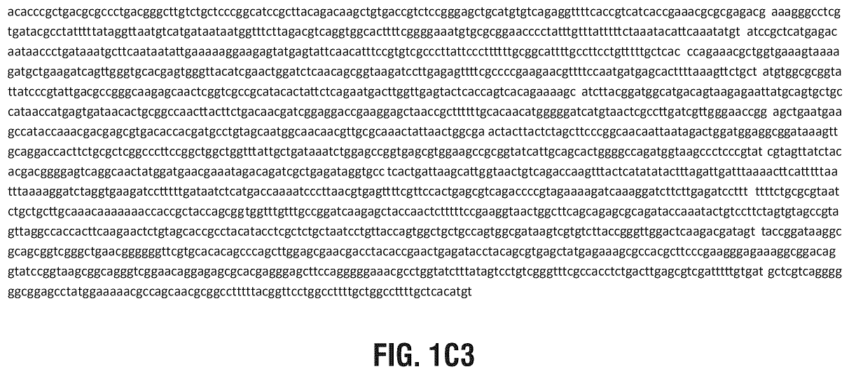

The guide RNA sequences were constructed as a pair of oligonucleotides which, once annealed, could be cloned into the BbsI sites of plasmid px458 (FIGS. 1A, 1B and 1C). This produced a single plasmid with a human U6-driven sgRNA sequence, followed by a CAG-driven Cas9 with a 2A-cleavable GFP. A list of plasmids is in Table 2, See FIG. 5.

TABLE 2

| | |
|---|---|
| pSL32 | oSL48/49 cloned into px458 |
| pSL33 | oSL50/51 cloned into px458 |
| pSL34 | oSL52/53 cloned into px458 |
| pSL35 | oSL54/55 cloned into px458 |
| pSL36 | oSL56/57 cloned into px458 |
| pSL37 | oSL58/59 cloned into px458 |
| pSL38 | oSL60/61 cloned into px458 |
| pSL39 | oSL64/65 cloned into px458 |
| pSL40 | oSL66/67 cloned into px458 |
| pSL41 | oSL68/69 cloned into px458 |
| pSL42 | oSL70/71 cloned into px458 |
| pSL43 | oSL72/73 cloned into px458 |
| pSL44 | oSL74/75 cloned into px458 |
| pSL45 | oSL76/77 cloned into px458 |
| pSL46 | oSL78/79 cloned into px458 |
| pSL47 | oSL80/81 cloned into px458 |
| pSL48 | oSL82/83 cloned into px458 |
| pSL49 | oSL62/63 cloned into px458 |

One microgram of plasmid miniprep DNA (Qiagen) was transfected into 6×10⁵ bovine embryonic fibroblast cells (BEF) using a Neon electroporator set at a single pulse of 1800 mV for 20 ms. Two days post transfection genomic DNA was prepared using the Qiagen DNeasy® Blood and Tissue kit. PCR was carried out on this genomic DNA using AccuPrime™ High Fidelity polymerase with PCR primers oSL868 and oSL87.

oSL868
(SEQ ID NO: 15)
agacgggtctttccagaggt
oSL87
(SEQ ID NO: 25)
acaagagtccggagagctga

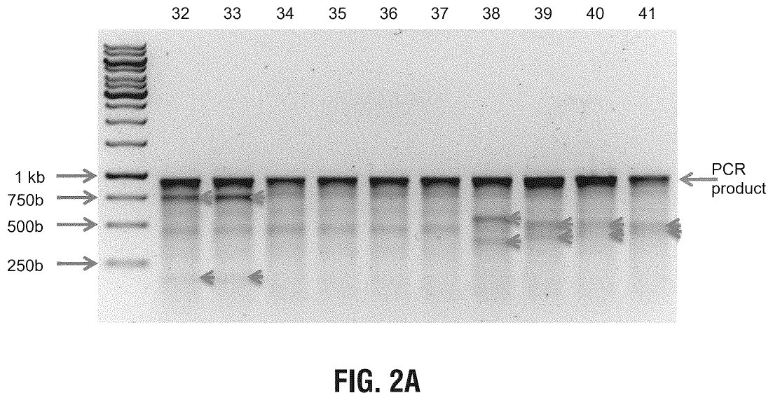

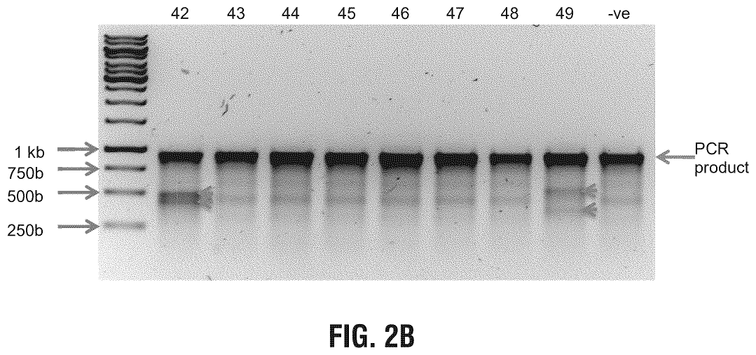

T7 endonuclease analysis was carried out on purified PCR products as recommended by the manufacturer (NEB). Digested PCR products were resolved on a 1.4% TAE agarose gel (FIGS. 2A and 2B). Surprisingly there were substantial differences discovered in the efficiencies with which sgRNAs were able to induce NHEJ formation at their target sites (indicated by the presence of T7 endonuclease digestion products, FIGS. 2A and 2B).

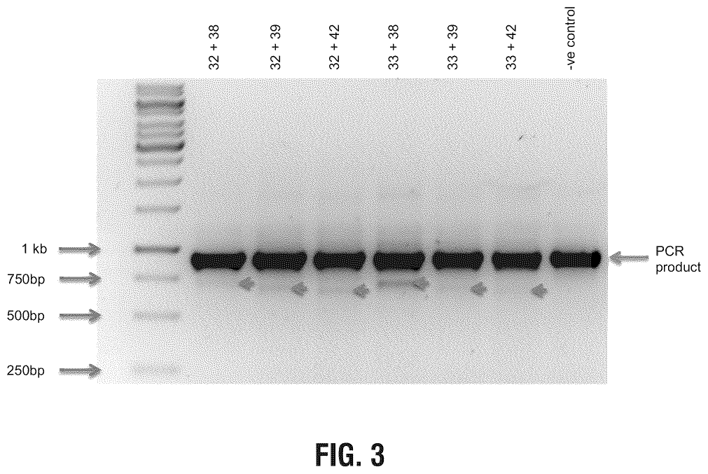

Guide sequences with the highest activity as determined by T7 endonuclease assay for NHEJ were combined in pairs, such that cleavage at both target sites within a cell has the potential to cause a deletion of the intervening sequence. One microgram each of plasmids pSL32+pSL38, pSL32+pSL39, pSL32+pSL42, pSL33+pSL38, pSL33+pSL39 or pSL33+pSL42 was transfected into 6×10⁵ BEF cells. Transfection, culturing and DNA preparation from BEF cells, was as above. PCR was carried out on genomic DNA using AccuPrime™ High Fidelity polymerase with PCR primers oSL868 and oSL87 and products were resolved on a 1.4% TAE agarose gel (FIG. 3). All six plasmid combinations demonstrated that, in addition to the full-length PCR product, transfected cell populations also harboured truncated NANOS2 gene fragments of a size specific to sgRNA sequence combination employed (Arrows, FIG. 3).

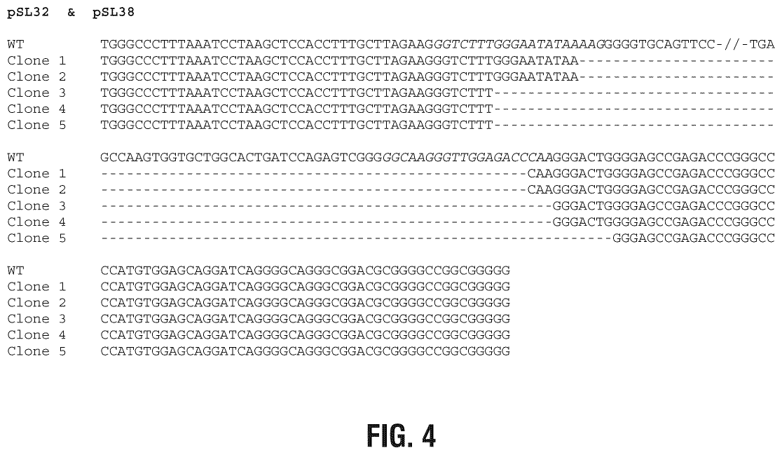

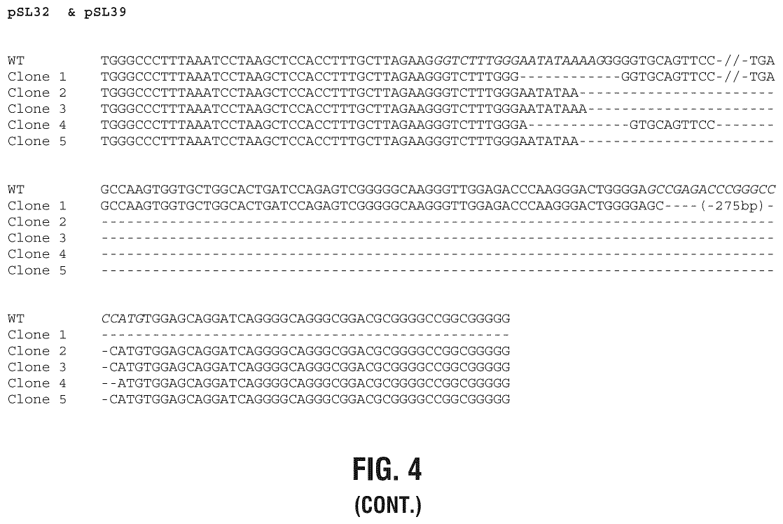

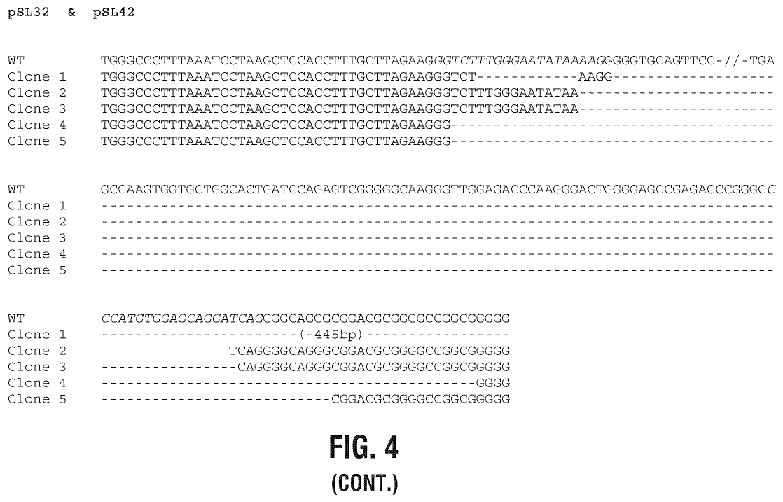

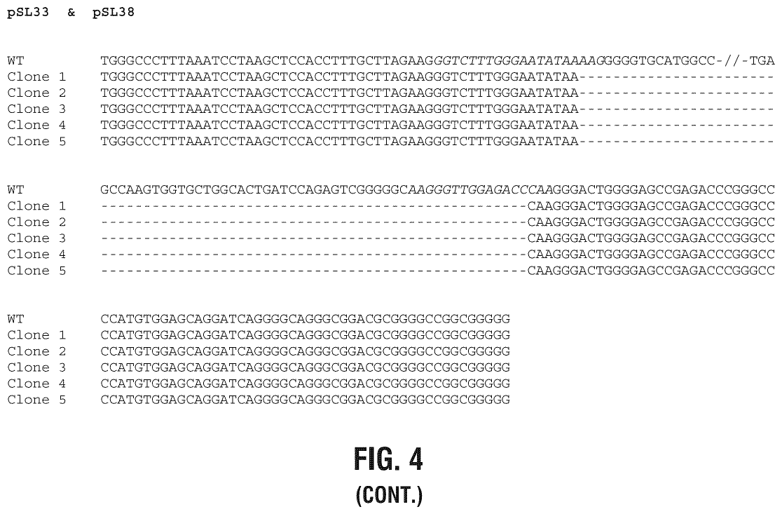

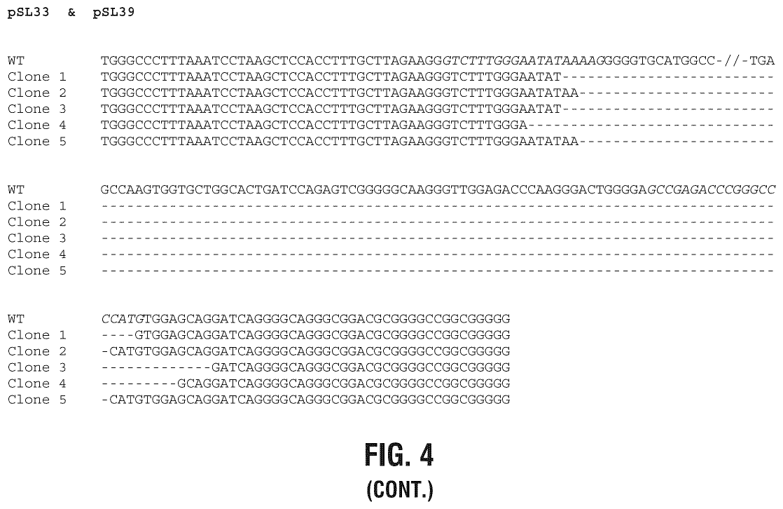

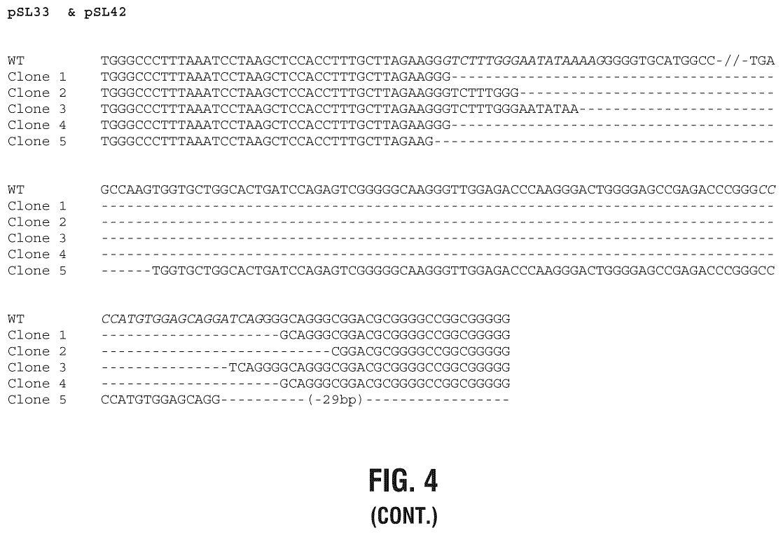

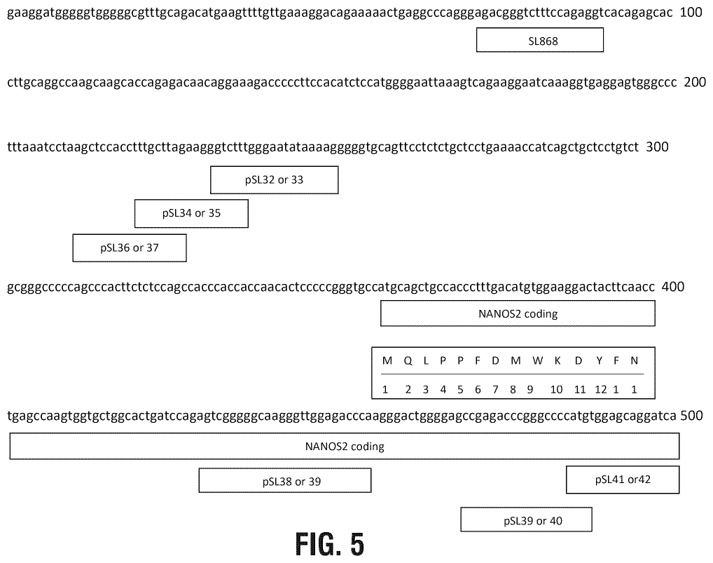

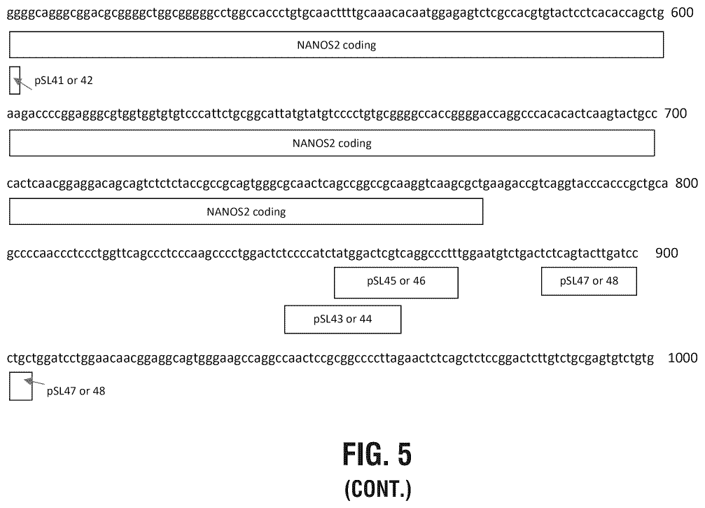

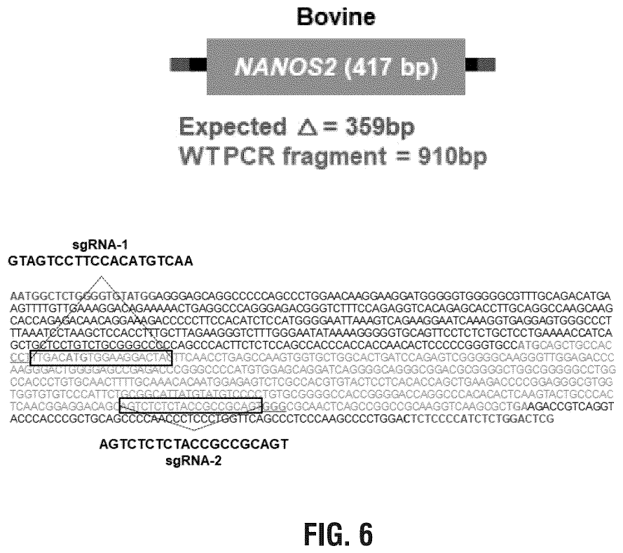

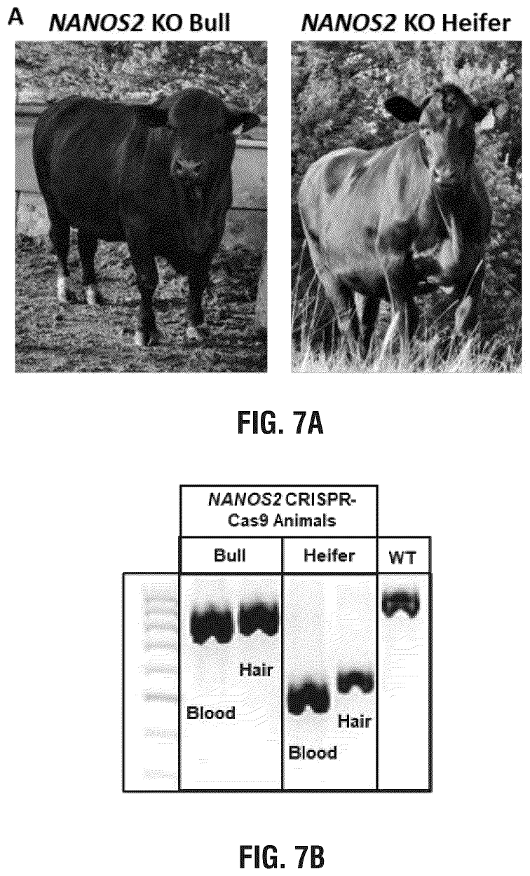

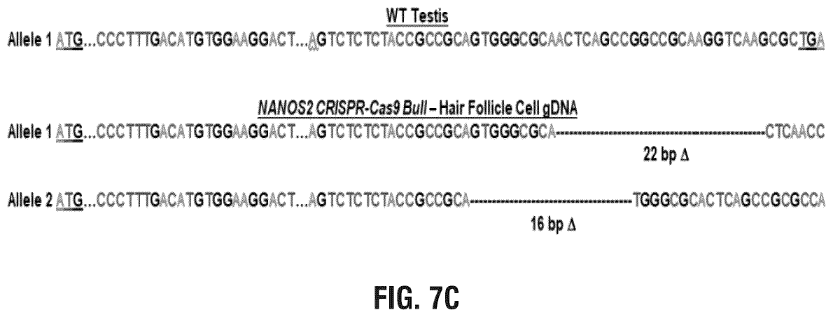

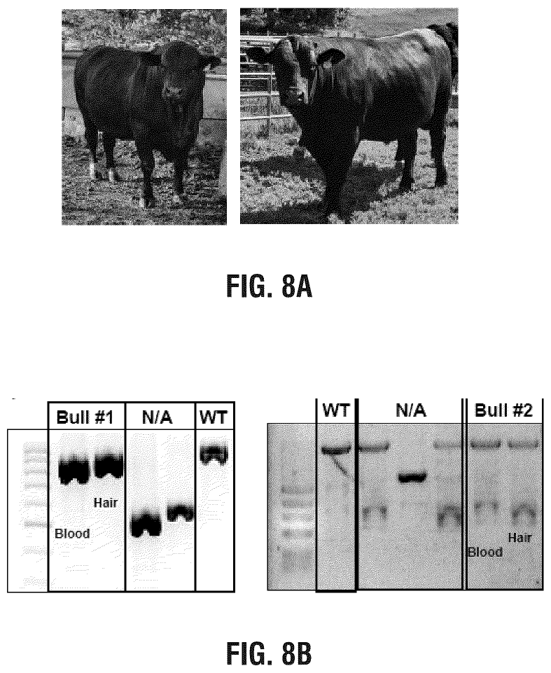

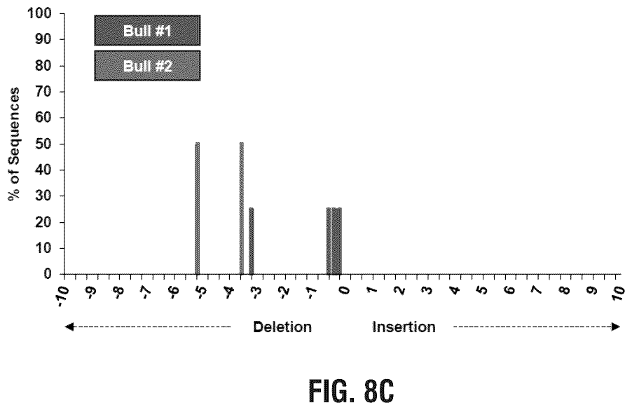

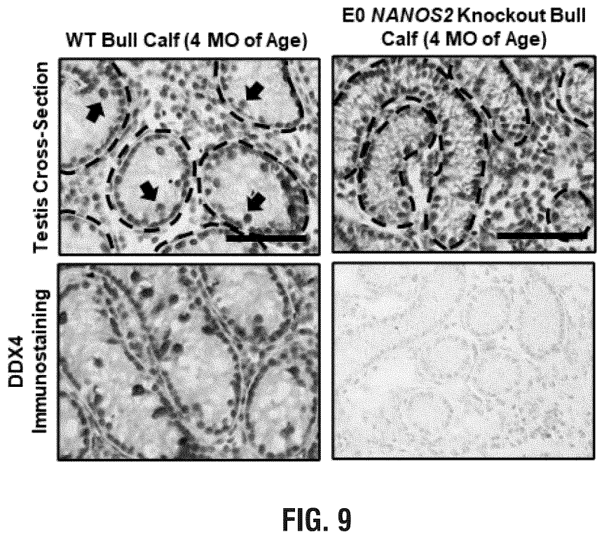

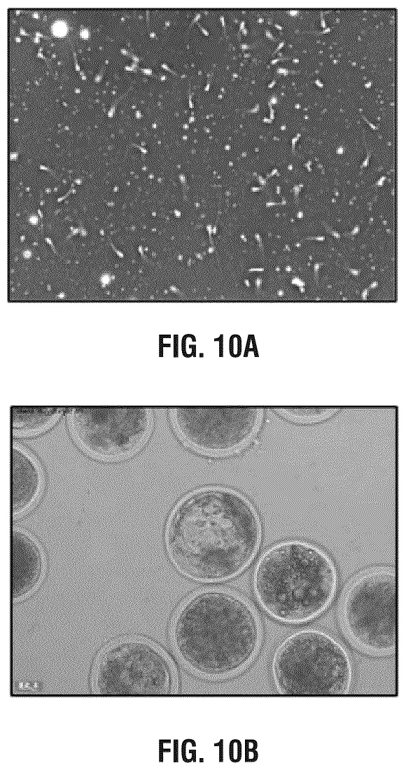

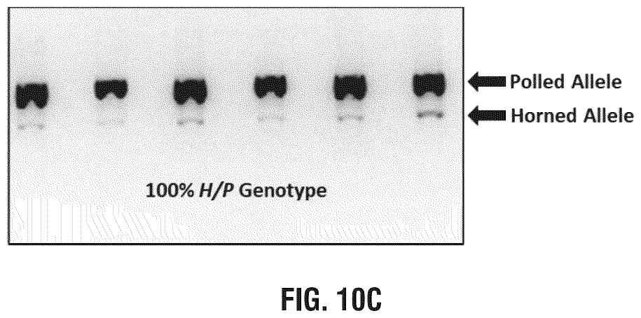

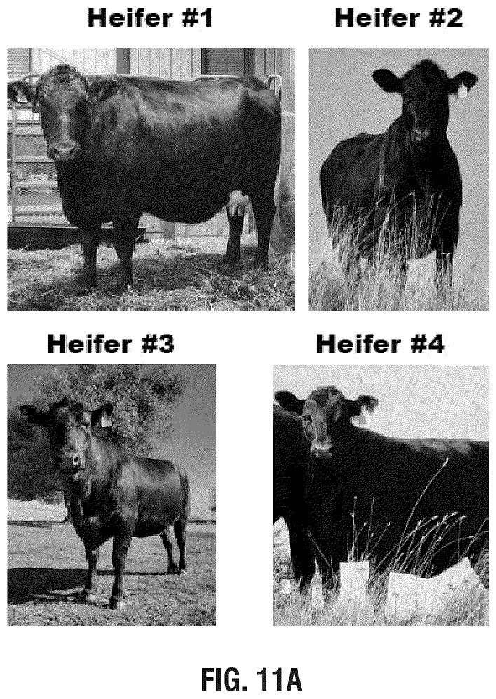

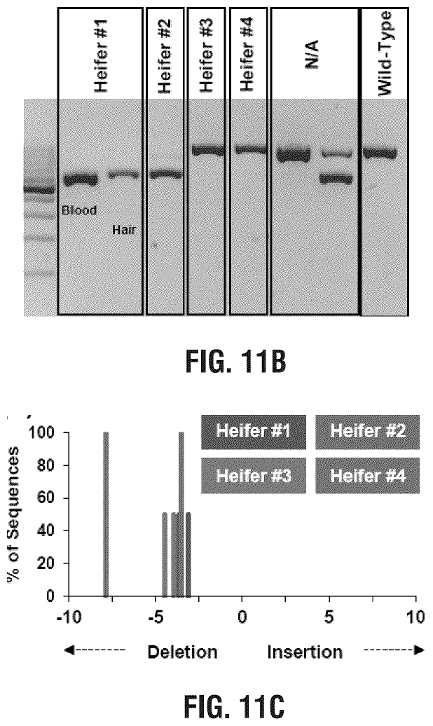

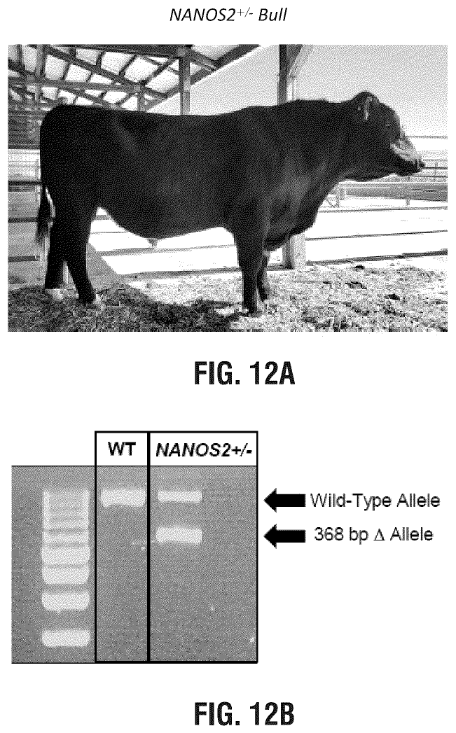

These truncated fragments were excised from the gel and DNA separated from the agarose by centrifuging the gel slice in a Spin-X® Centrifuge Tube Filter (Costar). The purified products were cloned into a pCR™ 4-TOPO™ vector using the TOPO® TA Cloning® Kit For Sequencing (Invitrogen) and transformed into XL-1 Blue Competent Cells (Agilent Technologies). Transformed bacteria were plated onto LB agar plates containing 100 μg/mL ampicillin and cultured at 37° C. overnight. Five colonies from each plate were selected and expanded overnight in LB medium containing 50 μg/mL ampicillin. Plasmid was isolated using a PureYield™ Plasmid Miniprep System (Promega) and sent for sequencing (Edinburgh Genomics) using primer oSL868. Alignment of the sequences demonstrates that deletion within the targeted sequence had occurred in each case (FIG. 4). Precise end joining between the ends of the deleted fragments was detected in at least one clone from each transfection, while other clones represented more imprecise events, indicating additional modification to the sequence occurred during the end-joining process. Clones sequenced from the pSL33+pSL38 combination all showed identical, precise end joining events.

This approach indicates that it is possible, and possibly desirable, to cause specific deletions in the target gene that result in loss or reduction of encoded protein expression or alteration of function of the resultant translated protein, as an additional strategy to the induction of NHEJ and consequent frame-shift mutations.

### Example 2

### CRISPR/Cas9 Reagent Generation for Bovine Embryos

Dual sgRNAs were designed to delete a large portion of the coding sequence (CDS) for the bovine NANOS2 gene. Briefly, candidate sgRNAs were designed and off-target predictor scores ranked using online software programs CRISPOR and CRISPR Design. The sense and antisense sgRNAs were designed to introduce a staggered double-stranded break that would result in deletion of a large portion of the CDS. To achieve this, sgRNAs were designed to target approximately 20 nucleotides upstream of the PAM sequences in the sense and antisense strand and adjacent to the start or stop codon. All selected sgRNAs had an off target predictor score of >80. The sgRNA sequences for targeting NANOS2 CDS are denoted in FIG. 6.

To generate sgRNAs, PCR reactions (using DreamTaq™; ThermoFischer Scientific) were used to create two overlapping oligonucleotides:
(1) CRISPRF that incorporates sequence specific for the desired target site, the 18 to 20 nucleotide guide sequence (N₂₅₋₄₅), and T7 promoter sequence:

(SEQ ID NO: 88)
GAAATTAATACGACTCACTATAGGN₂₅₋₄₅GTTTTAGAGCTAGAAATAGG;

and
(2) a common oligonucleotide that contains the sgRNA stem loop structure for docking of Cas9:

(SEQ ID NO: 89)
AAAAGCACCGACTCGGTGCCACTTTTTCAAGTTGATAACGGACTAGCCT
TATTTTAACTTGCTATTTCTAGCTCTAAAAC

The resultant dsDNA was gel purified (QIAquick Gel Purification Kit; Qiagen) and used as a template for synthesis of sgRNA with the MEGAshortscript™ Kit (ThermoFisher Scientific) and purified using the MEGAclear™ Kit (ThermoFisher Scientific) and eluted at approximately 2000 ng/μL in TE buffer (10 mM Tris-HCl, 0.1 mM EDTA, pH 7.4). sgRNAs targeting bovine NANOS2 were also procured from a commercial source (Synthego Inc.) For all sgRNAs, alliquots were stored at −80° C. for up to 6-months. For the generation of RNP complexes, sgRNAs (1000 ng/μL each) and Cas9 protein (200 ng/ml) sere diluted in TE buffer at a 1:1 mass ratio. Opti-MEM reduced Serum media with no phenol red (Gibco/ThermoFisher Scientific) was then added to provide a final reaction volume of 20 μL (containing 4000 ng of sgRNA and 4000 ng of Cas9 protein), and the mixture was incubated at room temperature for 10 min before being used as the electroporation solution. For bovine zygotes, TrueCut™ Cas9 protein v2 (ThermoFisher Scientific) was used.

### Example 3

### Generation of Bovine Zygotes and Embryo Culture

Bovine in vitro matured cumulus-oocytes complex (COCs) were washed and placed in 400 μL of equilibrated BO-IVF medium (IVF Biosciences) under embryo-safe mineral oil, in accordance with the IVF Biosciences protocol. Meanwhile, a ¼ cc straw of cryopreserved bovine semen was thawed at 37° C. for 30 seconds and viable sperm were subsequently isolated using a BoviPure™ gradient (Spectrum Technology, Healdsburg, CA, USA). Briefly, thawed sperm were overlaid on a 1 mL column of 80% BoviPure™ and centrifuged at 500×g for 15 min. The resulting sperm pellet was washed by centrifugation (300×g for 5 min) in TL-HEPES, and the pellet was suspended in equilibrated BO-IVF. At 20 to 24 h post onset of maturation, 50 μL of sperm suspension (standard concentration of 1× 10⁶ cells/mL) was added to 450 μL of IVF medium containing 50 matured oocytes and incubated at 38.5° C. in atmosphere of 5% CO₂ in air for about 18 h. Presumptive zygotes were denuded by vortexing in DPBS containing 0.1% hyaluronidase before being subjected to electroporation.

### Electroporation of Bovine Zygotes

At 18-22 h post IVF, zygotes (approximately 50-80) were washed with Opti-MEM solution and transferred to a 1 mm electroporation cuvette containing 20 μL of electroporation solution (sgRNA-Cas9 RNP complexes in Opti-MEM). Electroporation was carried out using an electroporation system (BTX T820 Square Wave Electroporation System). The optimal conditions were 2 pulses of 20 V/3 ms pulse length and 3 pulses of 30 V/3 ms pulse length for bovine and porcine zygotes, respectively. Following electroporation, embryos were recovered from the cuvette by washing with 1 mL TL-HEPES. Next, embryos were washed in TL-HEPES, then equilibrated BO-IVC 3×, and approximately 50 embryos were cultured to the blastocyst stage in 450 μL of equilibrated BO-IVC medium under embryo-safe mineral oil at 38.5° C. in an atmosphere of 5% CO₂ and 5% of 02. The 2-Cell and blastocyst rates were calculated based on the total number of zygotes electroporated at 3.5 and 7-8 days post fertilization, respectively.

### Genotyping of Embryos

To assess editing of the NANOS2 gene in bovine embryos, genomic DNA was obtained from a single embryo (2-Cell, 4-Cell, 8-Cell, or blastocyst). Embryos were suspended in 5 μL of lysis buffer (40 mM Tris, pH 8.9, 0.9% Triton X-100, 0.9% Nonidet P-40, 0.4 mg/mL proteinase K), incubated at 65° C. for 15 min, and heated to 95° C. for 10 min to inactivate the proteinase K. Reactions were neutralized by adding an equal volume of 40 mM Tris HCl (pH 5.5) buffer. For NANOS2 genotyping, PCR reactions were performed using 2×KAPA2G Fast HotStart® Genotyping Mix with dye (Roche, Switzerland) followed by cleanup with a QIAquick® Gel Purification kit (Qiagen). If some instances, DNA was ligated into PCR2.1 vectors (TACloning kit, Thermo Fisher Scientific), transformed into 10B E. coli (Zymo Research, Irvine, CA, USA), and 5 to 10 colonies were selected for analysis by PCR. All PCR products were visualized by agarose gel electrophoresis which allowed for detecting large (>100 bp) INDEL mutations. To assess smaller mutations and confirm large mutations, PCR products were subjected to sequencing analysis using the BigDye™ Terminator v3.1 Cycle Sequencing Kit (ThermoFisher Scientific) followed by cleanup (Mag-Bind™ SeqDTR, Omega Bio-Tek). Mutation detection was made by aligning base calls to published wild-type bovine NANOS2 DNA sequence.

TABLE 3
Bovine Embryos NANOS2 Genotyping (DNA Sequencing)

| | Embryo |
|---|---|
| Sequence (DNA) | Edits |
| AGCTGCCACCCTTTGACATGTGGAAGGACTACTTCAA...GACAGCAGTCTCTCTACCGCCGCAGTGGGC | WT, +/+ |
| GCAACTCAGCCG | |
| AGCT---ACC------ATGTAGGA----CTACTTCAA...GACAGCAGTGTGTGT----------TGGGC | bE1, Δ23 bp |
| GCAACTCAGCCG | |
| AGCT---ACC------ATGTAGGA----CTACTTCAA...GACAGCAGTGTGTGT----------TGGGC | bE1, Δ23 bp |
| GCAACTCAGCCG | |
| AGCTGCCACCCTTTGACATGTGGAAGGACTACTTCAA...-----------------------AGTGGGC | bE2, Δ32 bp |
| GCAACTCAGCCG | |
| -------------------------------------...------------------------------ | bE2, Δ390 bp |
| -CAACTCAGCCG | |
| GTAAGATGCCCTTTGACAT-TG-AAGGA-TA------...GACACGGGTCTCTCCTCT---GCAGTGGGC | bE3, Δ21 bp |
| GCAACTCAGCCG | |
| -------------------------------------...-----------------------AGTGGGC | bE3, Δ393 bp |
| GCAACTCAGCCG | |
| -----CCACCCTTTGACATGTGGAAGGACTACTTCAA...---------------------CCAGTGGGC | bE4, Δ34 bp |
| GCAACTCAGCCG | |
| AGCTGCCACCC--------------------------...-----------------------AGTGGGC | bE4, Δ363 bp |
| GCAA-TCAGCCG | |
| AGCTG---------------TGGAAGGACTACTTCAA...GACAGCAGTCTCTC---------------- | bE5, Δ46 bp |
| -----T-A-CC- | |
| -------------------------------------...------------------------------ | bE5, Δ387 bp |
| ------------ | |

### Generation of NANOS2 Gene Edited Animals

To generate NANOS2 gene edited cattle, zygote stage embryos electoroporated with ribonucleotide protein complexes of sgRNA-1-Cas9 and sgRNA-1-Cas9 (sgRNA sequence denoted in FIG. 6) were cultured to the early blastocyst stage (developmental day 6), and those that were scored as grades 1 to 2 were transferred into uterine horns of day 7- to 9-pseudopregnant recipient cows or heifers whose estrous cycles had been synchronized using the 5-day controlled internal drug release/cosynchronization protocol. For all recipients, two embryos were transferred into the uterine horn ipsolateral to an ovary possessing a robust corpus luteum using a standard nonsurgical technique. Pregnancy diagnosis was made at 60 days-postembryo transfer using ultrasonography. The transfers resulted in 6 pregnancies that produced live born offspring (FIGS. 7, 8, and 11). All live born calves were of normal birth weight and developed in a manner consistent with possessing normal physiology.

### Genotyping Analyses of NANOS2 Knock-Out Animals

Genomic DNA was isolated from blood cells, hair follicle cells, and skin epithelial cells of the six live born offspring using a DNeasy® kit (Qiagen). Genotyping for NANOS2 alleles in all samples was conducted using PCR and Sanger sequencing methodologies. PCR amplicon-based genotyping combined with Sanger sequencing analysis revealed that all live born calves possessed NANOS2 gene edits consistent with a knock-out genotype (FIGS. 7B, 7C, 8B, and 11B). In corroboration, tracking of INDELs by decomposition (TIDE) analysis for DNA sequencing outputs of the NANOS2 gene in CRISPR/Cas9 gene edited calves demonstrated an array of alleles that were inactivating (FIGS. 8C and 11C).

### Phenotyping of NANOS2 Knock-Out Bulls

To assess the biological ramifications of NANOS2 knock-out on the spermatogenic lineage, biopsies of testicular tissue were collected from the genomically edited bull calves at 3 to 4 months of age and fixed in Bouin's solution for 10 h. The tissue was then embedded in paraffin and 5 μm cross-sections generated for staining with hematoxylin and eosin. The morphology of the cross-sections was evaluated using light microscopy and digital images captured with Olympus IX51 microscope, Olympus TH4-100 digital camera, and Cell Sense Dimensions software. The images were captured with ×400 magnification. To determine presence or absence of germ cells, paraffin cross-sections (5 μm thickness) were adhered to glass slides, dewaxed, and rehydrated. The sections were then processed for antigen retrieval by incubating in an acidic (pH 6.0) solution of 10 mM sodium citrate (Amresco) and 0.05% Tween-20 (Sigma-Aldrich) for 20 min at 96° C. Nonspecific antibody binding was then blocked by incubating sections in a solution containing 2% bovine serum albumin (BSA) (Thermo Fisher Scientific) and 0.1% Triton X-100 (Sigma-Aldrich) in phosphate-buffered saline (PBS) at room temperature for 2 h. Sections were then incubated overnight at 4° C. with rabbit anti-human DDX4 primary antibody (Abcam) at 1:200 diution. Sections were washed in PBS and then incubated at room temperature for 2 h with a secondary antibody: biotinylated goat anti-rabbit IgG (Invitrogen) at 1:1,000 dilution. A horseradish peroxidase development reagent was then applied followed by washing in PBS, counterstaining with hematoxylin, and mounting cover-slips. Images were captured using an Olympus IX51 or Leica DMi8 microscope. Germ cells were clearly observable in cross-sections of testicular tissue from control wild-type bull calves but undetectable in cross-sections of testicular tissue from the NANOS2 CRISPR/Cas9 edited bull calves (FIG. 9). Although germ cells were absent, seminiferous tubules were intact with apparent somatic support cells (FIG. 9). Collectively, these findings solidify that NANOS2 is evolutionarily conserved as a specific regulator of male germline establishment and indicate that CRISPR/Cas9-based inactivation can produce germline ablated surrogate bulls.

### Example 4

### Fertility of NANOS2 Knock-Out Female Cattle

To assess the biological ramifications of NANOS2 knock-out on fertility of female cattle, the liveborn CRISPR/Cas9 edited heifers (FIG. 11) were first monitored via ultrasound imaging of the reproductive at 12 months of age. The assessments revealed the presence of ovarian follicles and corpus lutea thereby demonstrating normal ovarian function.

To determine fertility of NANOS2 knock-out female cattle, heifer #1 (FIG. 11A) who possessed a bi-allelic NANOS2 knock-out genotype (FIGS. 11B and 11C) was bred by artificial insemination with sperm from a wild-type bull. The heifer became pregnant and gave birth to a live bull calf (FIG. 12). Genotyping of the bull calf for NANOS2 revealed a heterozygous state with a wild-type intact allele and a deletion allele that was identical in size to the NANOS2 knock-out mother. This outcome provides unequivocal demonstration that NANOS2 knock-out female cattle are of normal fertility.

### Breeding for the Production of Homozygous NANOS2 Edited Males as SSC Recipients

A supply of homozygous NANOS2 edited males can be produced by breeding homozygous NANOS2 edited females with heterozygous edited males. The cross will produce equal numbers of homozygous and heterozygous edited males and females. Homozygous edited males will be used as SSC recipients, homozygous females and heterozygous males will be used as replacement breeding animals to produce further homozygous NANOS2 edited males as SSC recipients and heterozygous females will be culled.

### Example 5

### Spermatogenic Stem Cell Transplantation in NANOS2 Knock-Out Bulls

To determine if NANOS2 knock-out male cattle (bulls) can serve as surrogates for generation of sperm with the genetics of another male, spermatogenic stem cell transplantation was performed on a NANOS2 knock-out bull calf that was previously determined to be germline ablated (FIGS. 8 and 9). Donor spermatogenic stem cells (SSCs) were isolated from the testes of a wild-type bull and transferred into the testes of the NANOS2 knock-out bull at 4 months of age. At 8 months after transplantation, ejaculate samples from the NANOS2 knock-out recipient bull were collected and evaluated for sperm. A normal volume of ejaculate was produced which contained a concentration of viable sperm within the range of normal for a yearling beef breed bull at 1.5×10⁸/mL and a normal level of progressive motility at 89% and were found to be capable of fertilizing eggs in vitro (FIGS. 10A and 10B). To demonstrate that the sperm were of the SSC donor bull origin, in vitro fertilization was performed with eggs collected from cows that were homozygous for the Celtic polled allele (P/P). The NANOS2 knock-out bull possessed the genotype of heterozygous for polled and horned alleles (H/P); whereas the SSC donor male was homozygous for horned allele (H/H). By Mendelian inheritance laws, if the sperm were donor SSC derived, 100% of embryos would genotype as H/P. Indeed, the outcome of genotyping n=100 IVF embryos revealed that all were H/P (FIG. 10C). Collectively, these data demonstrate unequivocally that NANOS2 knock-out bulls have testicular somatic support cells that are functionally intact to support normal sperm production following transplantation of donor SSCs.

TABLE 4
NANOS Genes

NANOS 2 Bovine (Bos taurus)
NP_001268833.1
SEQ ID NO: 1
MQLPPFDMWKDYFNLSQVVLALIQSRGQGLETQGTGEPRPGPHVEQDQGQGGRGAG
GGLATLCNFCKHNGESRHVYSSHQLKTPEGVVVCPILRHYVCPLCGATGDQAHTLK
YCPLNGGQQSLYRRSGRNSAGRKVKR
NANOS 2 Bovine (Bos taurus)
NM_001281904.1
SEQ ID NO: 2

| | |
|---|---|
| 1 | tcagctgctc ctgtctgcgg gcccccagcc cacttctctc cagccaccca ccaccaacac |
| 61 | tcccccgggt gccatgcagc tgccaccctt tgacatgtgg aaggactact tcaacctgag |
| 121 | ccaagtggtg ctggcactga tccagagtcg ggggcaaggg ttggagaccc aagggactgg |
| 181 | ggagccgaga cccgggcccc atgtggagca ggatcagggg cagggcggac gcggggctgg |
| 241 | cgggggcctg gccaccctgt gcaacttttg caaacacaat ggagagtctc gccacgtgta |
| 301 | ctcctcacac cagctgaaga ccccggaggg cgtggtggtg tgtcccattc tgcggcatta |
| 361 | tgtatgtccc ctgtgcgggg ccaccgggga ccaggcccac acactcaagt actgcccact |
| 421 | caacggagga cagcagtctc tctaccgccg cagtgggcgc aactcagccg gccgcaaggt |
| 481 | caagcgctga agaccgtcag gtacccaccc gctgcagccc caaccctccc tggttcagcc |
| 541 | ctcccaag |

NANOS 1 Bovine (Bos taurus)
XP_005225853
SEQ ID NO: 3
MEAFPWAPRSPRRGRAPPPMALVPSARYVSTQGPAHPQPFSSWN
DYLGLATLITKAVDGEPRFGCARGGDGGGDGSPPSSSSSSCCSP
HVGAGPGALGPALGPPDYDEDDDDDDSDDPGSRSRYLGGALELR
ALELCADPAEAGLLEERFAELSPFAGRAAAVLLGCAPAAAAAAT
AEAAPREERAPAWAAEPKLHAASGAAAARLLKPELQVCVFCRNN
KEAVALYTTHILKGPDGRVLCPVLRRYTCPLCGASGDNAHTIKY
CPLSKVPPPPAARPPPRSARDGLPGKKLR
NANOS 1 Bovine (Bos taurus)
XM_005225796.5
SEQ ID NO: 4

| | |
|---|---|
| 1 | atggaggctt tcccctgggc gccccgctcg ccccgccgcg gccgcgcccc cccgcccatg |
| 61 | gcgctcgtgc ccagcgcccg ctacgtgagc acccagggcc cggcgcaccc gcagcccttc |
| 121 | agctcgtgga acgactatct gggactcgcc acgctcatca ccaaggcggt ggacggcgag |
| 181 | ccgcgcttcg gctgcgcccg cggcggggac ggcggcgggg acggctcccc gccttcttct |
| 241 | tcctcctcgt cgtgctgctc cccccacgtg ggggccgggc ctggggcgct ggggcccgcg |
| 301 | ctggggccgc ccgactacga cgaggacgac gacgacgacg acagcgacga tccggggtcc |
| 361 | cggagccgct acctgggggg cgcgctggag ctgcgcgcgc tggagctgtg cgcggaccct |
| 421 | gccgaggccg ggctgctgga ggagcgtttc gctgagctga gcccgttcgc tggtcgcgcc |
| 481 | gctgccgtgc ttctgggctg cgcacccgcc gccgccgccg cggccaccgc cgaagcagca |
| 541 | ccgcgagagg agcgggcccc ggcgtgggcg gccgagccca agctgcacgc cgcctccggg |
| 601 | gcggccgccg cccggctgct caagcccgag ctgcaggtgt gcgtgttttg ccggaacaac |
| 661 | aaggaggcgg tggcgctcta caccacccac atcctgaagg gacccgacgg gcgggtgctg |
| 721 | tgccccgtgc tgcgccggta cacgtgtccc ctgtgcggtg ccagcggcga caacgcgcac |
| 781 | accatcaagt actgcccgct ttccaaagtg ccgccgccgc ctgcagcccg cccgccgccg |
| 823 | cgcagcgccc gggacggcct gcccggcaag aagctgcgct aa |

NANOS 3 Bovine (Bos taurus)
XP_059744525.1
SEQ ID NO: 5
MWCVSVSCMCLCGPRCPYVCSGFYEQGCVAGAPCPTIPSAGFVS
GSPSHTLPCVCGPRAFVRVCSPCAFEVCVHLGSSVCPLAPPCPR
VPCAEAGSCVLESRGHPRLHPGAPGGRPRGTHGRGWTHRPAGAG
RWEGAVAAGSAGTSPRQTGGRVGGGGRGFEPAPEPRGPLPLPAAW
GAPAQPRSRQNAISRAGASRLGGLFRAGFGQKRGKQGGGPKTRFQ
RCQEVFWNSSLFLLPLNFCLRRLAWAG
NANOS 3 Bovine (Bos taurus)
XM_059888542.1
SEQ ID NO: 6

| | |
|---|---|
| 1 | atgtggtgcg tgtctgtatc ttgcatgtgt ctgtgtggcc ccaggtgccc ctacgtgtgt |
| 61 | agtgggtttt atgaacaagg ttgtgtggcg ggtgccccgt gtcctaccat tccctcggct |
| 121 | gggtttgtgt ctggaagtcc ctcgcacacc cttccctgtg tttgtggtcc gcgtgccttt |
| 181 | gtgcgcgtct gctccccgtg cgcctttgag gtctgcgtgc atctgggctc cagcgtgtgc |
| 241 | ccgctcgcgc ccccgtgtcc ccgtgtcccc tgcgcggagg ccgggagctg cgtcctggag |
| 301 | tcccgtgggc atccccgctt gcatcctggt gcgcctggcg ggaggcctcg gggcacacac |
| 361 | ggccgcggct ggacccaccg cccggcagga gccgggaggt gggagggggc ggtggcggcg |
| 421 | gggagcgcgg gcacgtcacc gagacagaca ggcgggcggg tgggaggcgg cggccgcggg |
| 481 | ttcgagccgg cgccggagcc ccgcggtccc ctccccctgc ccgcggcctg gggagccccc |
| 541 | gcccagcccc ggagccgcca aaatgcaatt tcccgtgccg gcgcctcgcg gctcgggggg |
| 601 | cttttccggg cgggttttgg acagaagagg gggaaacaag gcggcggccc caaaacgagg |
| 661 | ttccaaaggt gccaggaagt cttctggaac tcctccctct tcctgctgcc cctcaacttc |
| 721 | tgcctaagga gactggcgtg ggcaggatga |

All references cited herein are incorporated herein by reference in their entirety. Examples disclosed herein are provided by way of exemplification and are not intended to limit the scope of the invention.

TABLE OF SEQUENCES

| SEQ | TYPE | DESCRIPTION |
|---|---|---|
| SEQ ID NO: 1 | Protein | Bos taurus NANOS 2 |
| SEQ ID NO: 2 | DNA | Bos taurus NANOS 2 |
| SEQ ID NO: 3 | Protein | Bos taurus NANOS 1 |
| SEQ ID NO: 4 | DNA | Bos taurus NANOS 1 |
| SEQ ID NO: 5 | Protein | Bos taurus NANOS 3 |
| SEQ ID NO: 6 | DNA | Bos taurus NANOS 3 |
| SEQ ID NO: 7 (21) | DNA | FIG. 1B PX458 |
| SEQ ID NO: 8 (22) | DNA | FIG. 1B hu6 |
| SEQ ID NO: 9 (23) | RNA | FIGS. 1B gRNA scaffold |
| SEQ ID NO: 10 (24) | DNA | FIG. 1B terminal |
| SEQ ID NO: 11 (39) | DNA | FIG. 1C1 to 1C3 |
| SEQ ID NO: 12 (50) | DNA | FIG. 5 nt |
| SEQ ID NO: 13 (51) | Protein | FIG. 5 amino acid |
| SEQ ID NO: 14 (52) | DNA | FIG. 5 NANOS nt CDS |
| SEQ ID NO: 15 (64) | nucleotide | FIG. 5 oSL868 |
| SEQ ID NO: 16 (65) | nucleotide | FIG. 5 pSL36 or 37 |
| SEQ ID NO: 17 (66) | nucleotide | FIG. 5 pSL34 or 35 |
| SEQ ID NO: 18 (67) | nucleotide | FIG. 5 pSL32 or 33 |
| SEQ ID NO: 19 (68) | nucleotide | FIG. 5 pSL38 or 39 |
| SEQ ID NO: 20 (69) | nucleotide | FIG. 5 pSL39 or 40 |
| SEQ ID NO: 21 (70) | nucleotide | FIG. 5 pSL41 or 42 |
| SEQ ID NO: 22 (71) | nucleotide | FIG. 5 pSL43 or 44 |
| SEQ ID NO: 23 (72) | nucleotide | FIG. 5 pSL45 or 46 |
| SEQ IN NO: 24 (73) | nucleotide | FIG. 5 pSL47 or 48 |
| SEQ ID NO: 25 (74) | nucleotide | FIG. 5 oSL87 |
| SEQ ID NO: 26 (88) | nucleotide | oSL48 Table 1 |
| SEQ ID NO: 27 (89) | nucleotide | oSL49 Table 1 |
| SEQ ID NO: 28 (90) | nucleotide | oSL50 Table 1 |
| SEQ ID NO: 29 (91) | nucleotide | oSL51 Table 1 |
| SEQ ID NO: 30 (92) | nucleotide | oSL52 Table 1 |
| SEQ ID NO: 31 (93) | nucleotide | oSL53 Table 1 |
| SEQ ID NO: 32 (94) | nucleotide | oSL54 Table 1 |
| SEQ ID NO: 33 (95) | nucleotide | oSL55 Table 1 |
| SEQ ID NO: 34 (96) | nucleotide | oSL56 Table 1 |
| SEQ ID NO: 35 (97) | nucleotide | oSL57 Table 1 |
| SEQ ID NO: 36 (98) | nucleotide | oSL58 Table 1 |
| SEQ ID NO: 37 (99) | nucleotide | oSL59 Table 1 |
| SEQ IN NO: 38 (100) | nucleotide | oSL60 Table 1 |
| SEQ ID NO: 39 (101) | nucleotide | oSL61 Table 1 |
| SEQ ID NO: 40 (102) | nucleotide | oSL62 Table 1 |
| SEQ ID NO: 41 (103) | nucleotide | oSL63 Table 1 |
| SEQ ID NO: 42 (104) | nucleotide | oSL64 Table 1 |
| SEQ ID NO: 43 (105) | nucleotide | oSL65 Table 1 |
| SEQ ID NO: 44 (106) | nucleotide | oSL66 Table 1 |
| SEQ ID NO: 45 (107) | nucleotide | oSL67 Table 1 |
| SEQ ID NO: 46 (108) | nucleotide | oSL68 Table 1 |
| SEQ ID NO: 47 (109) | nucleotide | oSL69 Table 1 |
| SEQ ID NO: 48 (110) | nucleotide | oSL70 Table 1 |
| SEQ ID NO: 49 (111) | nucleotide | oSL71 Table 1 |
| SEQ ID NO: 50 (112) | nucleotide | oSL72 Table 1 |
| SEQ ID NO: 51 (113) | nucleotide | oSL73 Table 1 |
| SEQ ID NO: 52 (114) | nucleotide | oSL74 Table 1 |
| SEQ ID NO: 53 (115) | nucleotide | oSL75 Table 1 |
| SEQ ID NO: 54 (116) | nucleotide | oSL76 Table 1 |
| SEQ ID NO: 55 (117) | nucleotide | pSL32 & pSL38: WT FIG. 4 |
| SEQ ID NO: 56 (118) | nucleotide | pSL32 & pSL38 Clone 1 FIG. 4 |
| SEQ ID NO: 56 (119) | nucleotide | pSL32 & pSL38 Clone 2 FIG. 4 |
| SEQ ID NO: 57 (120) | nucleotide | pSL32 & pSL38 Clone 3 FIG. 4 |
| SEQ ID NO: 57 (121) | nucleotide | pSL32 & pSL38 Clone 4 FIG. 4 |
| SEQ ID NO: 58 (122) | nucleotide | pSL32 & pSL38 Clone 5 FIG. 4 |
| SEQ IDNO: 55 (123) | nucleotide | pSL32 & pSL39: WT FIG. 4 |
| SEQ IDNO: 59 (124) | nucleotide | pSL32 & pSL39: Clone 1 FIG. 4 |
| SEQ IDNO: 60 (125) | nucleotide | pSL32 & pSL39: Clone 2 FIG. 4 |
| SEQ IDNO: 61 (126) | nucleotide | pSL32 & pSL39: Clone 3 FIG. 4 |
| SEQ IDNO: 62 (127) | nucleotide | pSL32 & pSL39: Clone 4 FIG. 4 |
| SEQ IDNO: 60 (128) | nucleotide | pSL32 & pSL39: Clone 5 FIG. 4 |
| SEQ ID NO: 55 (129) | nucleotide | pSL32 & pSL42 WT FIG. 4 |
| SEQ ID NO: 63 (130) | nucleotide | pSL32 & pSL42 Clone 1 FIG. 4 |
| SEQ ID NO: 64 (131) | nucleotide | pSL32 & pSL42 Clone 2 FIG. 4 |
| SEQ ID NO: 65 (132) | nucleotide | pSL32 & pSL42 Clone 3 FIG. 4 |
| SEQ ID NO: 66 (133) | nucleotide | pSL32 & pSL42 Clone 4 FIG. 4 |
| SEQ ID NO: 67 (134) | nucleotide | pSL32 & pSL42 Clone 5 FIG. 4 |
| SEQ ID NO: 55 (135) | nucleotide | pSL33 & pSL38 WT FIG. 4 |
| SEQ ID NO: 68 (136) | nucleotide | pSL33 & pSL38 Clone 1 FIG. 4 |
| SEQ ID NO: 68 (137) | nucleotide | pSL33 & pSL38 Clone 2FIG. 4 |
| SEQ ID NO: 68 (138) | nucleotide | pSL33 & pSL38 Clone 3 FIG. 4 |
| SEQ ID NO: 68 (139) | nucleotide | pSL33 & pSL38 Clone 4 FIG. 4 |
| SEQ ID NO: 68 (140) | nucleotide | pSL33 & pSL38 Clone 5 FIG. 4 |
| SEQ ID NO: 55 (141) | nucleotide | pSL33 & pSL39 WT FIG. 4 |
| SEQ ID NO: 69 (142) | nucleotide | pSL33 & pSL39 Clone 1 FIG. 4 |
| SEQ ID NO: 70 (143) | nucleotide | pSL33 & pSL39 Clone 2 FIG. 4 |
| SEQ ID NO: 71 (144) | nucleotide | pSL33 & pSL39 Clone 3 FIG. 4 |
| SEQ ID NO: 72 (145) | nucleotide | pSL33 & pSL39 Clone 4 FIG. 4 |
| SEQ ID NO: 73 (146) | nucleotide | pSL33 & pSL39 Clone 5 FIG. 4 |
| SEQ ID NO: 55 (147) | nucleotide | pSL33 & pSL42 WT FIG. 4 |
| SEQ ID NO: 74 (148) | nucleotide | pSL33 & pSL42 Clone 1 FIG. 4 |
| SEQ ID NO: 75 (149) | nucleotide | pSL33 & pSL42 Clone 2 FIG. 4 |
| SEQ ID NO: 76 (150) | nucleotide | pSL33 & pSL42 Clone 3 FIG. 4 |
| SEQ ID NO: 75 (151) | nucleotide | pSL33 & pSL42 Clone 4 FIG. 4 |
| SEQ ID NO: 77 (152) | nucleotide | pSL33 & pSL42 Clone 5 FIG. 4 |
| SEQ ID NO: 78 (153) | nucleotide | oSL77 Table 1 |
| SEQ ID NO: 79 (154) | nucleotide | oSL78 Table 1 |
| SEQ ID NO: 80 (155) | nucleotide | oSL79 Table 1 |
| SEQ ID NO: 81 (156) | nucleotide | oSL80 Table 1 |
| SEQ ID NO: 82 (157) | nucleotide | oSL81 Table 1 |
| SEQ ID NO: 83 (158) | nucleotide | oSL82 Table 1 |
| SEQ ID NO: 84 (159) | nucleotide | oSL83 Table 1 |
| SEQ ID NO: 85 | RNA | sgRNA-1 FIG. 6 |
| SEQ ID NO: 86 | RNA | sgRNA-2 FIG. 6 |
| SEQ ID NO: 87 | DNA | FIG. 6 |
| SEQ ID NO: 88 | DNA | CRISPRF Example 2 |
| SEQ ID NO: 89 | RNA | oligonucleotide w/sgRNA stem loop |
| | | Example 2 |

1. A genetically edited bovine animal comprising at least one edited chromosomal sequence encoding a NANOS2 protein.

2. The genetically edited bovine animal of claim 1, wherein the edited chromosomal sequence is inactivated, modified, and/or comprises an integrated or deleted sequence.

3. The genetically edited bovine animal of claim 1, wherein the edited chromosomal sequence is inactivated such that little or no functional NANOS2 protein activity is produced and said animal produces reduced or no germline cells but retains testicular somatic cell function.

4. The genetically edited bovine animal of claim 1, wherein the edited chromosomal sequence comprises no exogenously introduced sequence.

5. The genetically edited bovine animal of claim 1 further comprising a conditional knock-out system for conditional expression of the NANOS2 protein.

6. The genetically edited bovine animal of claim 1, wherein the edited chromosomal sequence comprises an integrated reporter sequence.

7. The genetically edited bovine animal of claim 1, wherein the animal is heterozygous or homozygous for at least one edited chromosomal sequence.

8. The genetically edited bovine animal of claim 7, wherein said animal includes an edited NANOS2 gene of SEQ ID NO:50, 51, 52, 56, 57, 58, 59, 60, 61, 62, 63, 64, 65, 66, 67, 68, 69, 70, 71, 72, 73, 74, 75, 76, 77, 91, 92, 93, 94, 95, 96, 97, or 98.

9. The genetically edited bovine animal of claim 1, further comprising a recipient of donor spermatogenic stem cells (SSCs) or cells that can give rise to cells of the spermatogenic lineage to create a surrogate male bovine sire.

10. The recipient germ line ablated surrogate male bovine sire of claim 9.

11. Sperm from the recipient surrogate male bovine of claim 10.

12. A genetically edited bovine animal cell from the genetically edited bovine animal of claim 1, wherein the cell comprises at least one edited chromosomal sequence encoding a NANOS2 protein.

13. The genetically edited bovine animal of claim 12 wherein the edited chromosomal sequence is inactivated, modified, or comprises an integrated sequence.

14. The genetically edited bovine animal of claim 12, wherein the edited chromosomal sequence is inactivated such that the NANOS2 protein function is modulated.

15. The genetically edited bovine animal of claim 12, wherein the cell is heterozygous or homozygous for the at least one edited chromosomal sequence.

16. The genetically edited bovine animal of claim 12, wherein said cell includes an edited NANOS2 gene of SEQ ID NO:50, 51, 52, 56, 57, 58, 59, 60, 61, 62, 63, 64, 65, 66, 67, 68, 69, 70, 71, 72, 73, 74, 75, 76, 77, 91, 92, 93, 94, 95, 96, 97, or 98.

17. The genetically edited bovine animal of claim 15, wherein the inactivated chromosomal sequence comprises no exogenously introduced sequence.

18. The genetically edited bovine animal of claim 12, further comprising a conditional knock-out system for conditional expression of the NANOS2 protein.

19. The genetically edited bovine animal cell of claim 13, wherein the edited chromosomal sequence comprises an integrated reporter sequence.

20. A method of breeding bovine animals comprising:
collecting sperm from the germ line ablated surrogate male recipient of claim 17 and introducing said sperm into a female animal so that said female animal becomes pregnant.

21. A bovine animal produced by the method of claim 20.

22. The method of claim 20, wherein said introducing is by artificial insemination.

23. A method of generating a bovine animal with no functional spermatogonial cells comprising:
modifying NANOS2 gene expression so that NANOS2 protein function is reduced or eliminated.

24. The method of claim 23, wherein said bovine animal does not produce a significant amount of spermatogonial cells.

25. The method of claim 23, wherein said modifying is by gene editing using a TALEN, a zinc finger nuclease, CRISPR/Cas9, and/or a recombinase fusion protein.

26. The method of claim 23, wherein said NANOS2 gene is edited to include an insertion or deletion which causes inactivation of the gene.

27. The method of claim 25, wherein said gene editing modification step includes the use of a NANOS2 guide RNA, and a polypeptide capable of affecting cleavage or integration of the NANOS2 target gene.

28. A method of producing a recipient bovine male animal that serves as a surrogate for donor-derived sperm production for transmission of the donor genotype to offspring via natural or artificial breeding comprising:
collecting donor SSCs from a desired male donor, and thereafter,
transplanting donor SSCs to a NANOS2 knock-out (−/−) recipient male so that spermatogenic colonies are generated.

29. A method of producing bovine animals comprising:
mating a female bovine animal with sperm from the recipient NANOS2 knock-out (−/−) male of claim 21.

30. A method to increase availability of sperm from a desirable male mammal, the method comprising:
a. producing a genetically edited recipient male mammal that serves as a surrogate for donor-derived sperm production, wherein the recipient male mammal has a bi-allelic loss-of-function NANOS2 knock-out (−/−);
b. collecting spermatogenic stem cells (SSCs) or cells capable of giving rise to cells of the spermatogenic lineage from the desirable donor mammal;
c. implanting the donor SSCs or cells capable of giving rise to cells of the spermatogenic lineage into the recipient male mammal, wherein the SSCs or cells capable of giving rise to cells of the spermatogenic lineage engraft or colonize within the seminiferous tubules/cords of the testes of the recipient male mammal;
d. collecting sperm from the recipient male mammal; and
e. inseminating a female mammal with the sperm from the recipient male mammal to produce progeny comprising genetic characteristics of the desirable male mammal.


SEQUENCE LISTING (101 sequences)

SEQ ID NO: 1 - protein, 138 aa, Bos taurus
MQLPPFDMWKDYFNLSQVVLALIQSRGQGLETQGTGEPRPGPHVEQDQGQGGRGAGGGLATLCNFCKHNGESRHVYSSHQLKTPEGVVVCPILRHYVCPLCGATGDQAHTLKYCPLNGGQQSLYRRSGRNSAGRKVKR

SEQ ID NO: 2 - DNA, 548 bp, Bos taurus
  source 1..548: mol_type = other DNA
tcagctgctcctgtctgcgggcccccagcccacttctctccagccacccaccaccaacactcccccgggtgccatgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcaactcagccggccgcaaggtcaagcgctgaagaccgtcaggtacccacccgctgcagccccaaccctccctggttcagccctcccaag

SEQ ID NO: 3 - protein, 293 aa, Bos taurus
MEAFPWAPRSPRRGRAPPPMALVPSARYVSTQGPAHPQPFSSWNDYLGLATLITKAVDGEPRFGCARGGDGGGDGSPPSSSSSSCCSPHVGAGPGALGPALGPPDYDEDDDDDDSDDPGSRSRYLGGALELRALELCADPAEAGLLEERFAELSPFAGRAAAVLLGCAPAAAAAATAEAAPREERAPAWAAEPKLHAASGAAAARLLKPELQVCVFCRNNKEAVALYTTHILKGPDGRVLCPVLRRYTCPLCGASGDNAHTIKYCPLSKVPPPPAARPPPRSARDGLPGKKLR

SEQ ID NO: 4 - DNA, 882 bp, Bos taurus
  source 1..882: mol_type = other DNA
atggaggctttcccctgggcgccccgctcgccccgccgcggccgcgcccccccgcccatggcgctcgtgcccagcgcccgctacgtgagcacccagggcccggcgcacccgcagcccttcagctcgtggaacgactatctgggactcgccacgctcatcaccaaggcggtggacggcgagccgcgcttcggctgcgcccgcggcggggacggcggcggggacggctccccgccttcttcttcctcctcgtcgtgctgctccccccacgtgggggccgggcctggggcgctggggcccgcgctggggccgcccgactacgacgaggacgacgacgacgacgacagcgacgatccggggtcccggagccgctacctggggggcgcgctggagctgcgcgcgctggagctgtgcgcggaccctgccgaggccgggctgctggaggagcgtttcgctgagctgagcccgttcgctggtcgcgccgctgccgtgcttctgggctgcgcacccgccgccgccgccgcggccaccgccgaagcagcaccgcgagaggagcgggccccggcgtgggcggccgagcccaagctgcacgccgcctccggggcggccgccgcccggctgctcaagcccgagctgcaggtgtgcgtgttttgccggaacaacaaggaggcggtggcgctctacaccacccacatcctgaagggacccgacgggcgggtgctgtgccccgtgctgcgccggtacacgtgtcccctgtgcggtgccagcggcgacaacgcgcacaccatcaagtactgcccgctttccaaagtgccgccgccgcctgcagcccgcccgccgccgcgcagcgcccgggacggcctgcccggcaagaagctgcgctaa

SEQ ID NO: 5 - protein, 249 aa, Bos taurus
MWCVSVSCMCLCGPRCPYVCSGFYEQGCVAGAPCPTIPSAGFVSGSPSHTLPCVCGPRAFVRVCSPCAFEVCVHLGSSVCPLAPPCPRVPCAEAGSCVLESRGHPRLHPGAPGGRPRGTHGRGWTHRPAGAGRWEGAVAAGSAGTSPRQTGGRVGGGGRGFEPAPEPRGPLPLPAAWGAPAQPRSRQNAISRAGASRLGGLFRAGFGQKRGKQGGGPKTRFQRCQEVFWNSSLFLLPLNFCLRRLAWAG

SEQ ID NO: 6 - DNA, 750 bp, Bos taurus
  source 1..750: mol_type = other DNA
atgtggtgcgtgtctgtatcttgcatgtgtctgtgtggccccaggtgcccctacgtgtgtagtgggttttatgaacaaggttgtgtggcgggtgccccgtgtcctaccattccctcggctgggtttgtgtctggaagtccctcgcacacccttccctgtgtttgtggtccgcgtgcctttgtgcgcgtctgctccccgtgcgcctttgaggtctgcgtgcatctgggctccagcgtgtgcccgctcgcgcccccgtgtccccgtgtcccctgcgcggaggccgggagctgcgtcctggagtcccgtgggcatccccgcttgcatcctggtgcgcctggcgggaggcctcggggcacacacggccgcggctggacccaccgcccggcaggagccgggaggtgggagggggcggtggcggcggggagcgcgggcacgtcaccgagacagacaggcgggcgggtgggaggcggcggccgcgggttcgagccggcgccggagccccgcggtcccctccccctgcccgcggcctggggagcccccgcccagccccggagccgccaaaatgcaatttcccgtgccggcgcctcgcggctcggggggcttttccgggcgggttttggacagaagagggggaaacaaggcggcggccccaaaacgaggttccaaaggtgccaggaagtcttctggaactcctccctcttcctgctgcccctcaacttctgcctaaggagactggcgtgggcaggatga

SEQ ID NO: 7 - DNA, 360 bp, synthetic construct
  source 1..360: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagct

SEQ ID NO: 8 - DNA, 240 bp, synthetic construct
  source 1..240: mol_type = other DNA
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 9 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 10 - skipped

SEQ ID NO: 11 - DNA, 9289 bp, synthetic construct
  source 1..9289: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcaatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaactttttttcaggttggaccggtgccaccatggactataaggaccacgacggagactacaaggatcatgatattgattacaaagacgaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 12 - DNA, 1000 bp, Bos taurus
  source 1..1000: mol_type = other DNA
gaaggatgggggtgggggcgtttgcagacatgaagttttgttgaaaggacagaaaaactgaggcccagggagacgggtctttccagaggtcacagagcaccttgcaggccaagcaagcaccagagacaacaggaaagacccccttccacatctccatggggaattaaagtcagaaggaatcaaaggtgaggagtgggccctttaaatcctaagctccacctttgcttagaagggtctttgggaatataaaagggggtgcagttcctctctgctcctgaaaaccatcagctgctcctgtctgcgggcccccagcccacttctctccagccacccaccaccaacactcccccgggtgccatgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcaactcagccggccgcaaggtcaagcgctgaagaccgtcaggtacccacccgctgcagccccaaccctccctggttcagccctcccaagcccctggactctccccatctatggactcgtcaggccctttggaatgtctgactctcagtacttgatccctgctggatcctggaacaacggaggcagtgggaagccaggccaactccgcggccccttagaactctcagctctccggactcttgtctgcgagtgtctgtg

SEQ ID NO: 13 - protein, 14 aa, Bos taurus
MQLPPFDMWKDYFN

SEQ ID NO: 14 - DNA, 414 bp, Bos taurus
  source 1..414: mol_type = other DNA
atgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcaactcagccggccgcaaggtcaagcgc

SEQ ID NO: 15 - DNA, 20 bp, synthetic construct
  source 1..20: mol_type = other DNA
agacgggtctttccagaggt

SEQ ID NO: 16 - RNA, 20 nt, synthetic construct
  source 1..20: mol_type = other RNA
aagctccacctttgcttaga

SEQ ID NO: 17 - RNA, 20 nt, synthetic construct
  source 1..20: mol_type = other RNA
ctttgcttagaagggtcttt

SEQ ID NO: 18 - RNA, 20 nt, synthetic construct
  source 1..20: mol_type = other RNA
ggtctttgggaatataaaag

SEQ ID NO: 19 - RNA, 19 nt, synthetic construct
  source 1..19: mol_type = other RNA
ggcaagggttggagaccca

SEQ ID NO: 20 - RNA, 20 nt, synthetic construct
  source 1..20: mol_type = other RNA
gccgagacccgggccccatg

SEQ ID NO: 21 - RNA, 20 nt, synthetic construct
  source 1..20: mol_type = other RNA
cccatgtggagcaggatcag

SEQ ID NO: 22 - RNA, 19 nt, synthetic construct
  source 1..19: mol_type = other RNA
tccccatctatggactcgt

SEQ ID NO: 23 - RNA, 20 nt, synthetic construct
  source 1..20: mol_type = other RNA
tatggactcgtcaggccctt

SEQ ID NO: 24 - RNA, 20 nt, synthetic construct
  source 1..20: mol_type = other RNA
tctcagtacttgatccctgc

SEQ ID NO: 25 - RNA, 20 nt, synthetic construct
  source 1..20: mol_type = other RNA
acaagagtccggagagctga

SEQ ID NO: 26 - RNA, 24 nt, synthetic construct
  source 1..24: mol_type = other RNA
caccggtctttgggaatataaaag

SEQ ID NO: 27 - RNA, 24 nt, synthetic construct
  source 1..24: mol_type = other RNA
aaaccttttatattcccaaagacc

SEQ ID NO: 28 - RNA, 23 nt, synthetic construct
  source 1..23: mol_type = other RNA
caccgtctttgggaatataaaag

SEQ ID NO: 29 - RNA, 23 nt, synthetic construct
  source 1..23: mol_type = other RNA
aaaccttttatattcccaaagac

SEQ ID NO: 30 - RNA, 25 nt, synthetic construct
  source 1..25: mol_type = other RNA
caccgctttgcttagaagggtcttt

SEQ ID NO: 31 - RNA, 25 nt, synthetic construct
  source 1..25: mol_type = other RNA
aaacaaagacccttctaagcaaagc

SEQ ID NO: 32 - RNA, 23 nt, synthetic construct
  source 1..23: mol_type = other RNA
caccgttgcttagaagggtcttt

SEQ ID NO: 33 - RNA, 23 nt, synthetic construct
  source 1..23: mol_type = other RNA
aaacaaagacccttctaagcaac

SEQ ID NO: 34 - RNA, 25 nt, synthetic construct
  source 1..25: mol_type = other RNA
caccgagctccacctttgcttagaa

SEQ ID NO: 35 - RNA, 25 nt, synthetic construct
  source 1..25: mol_type = other RNA
aaacttctaagcaaaggtggagctc

SEQ ID NO: 36 - RNA, 23 nt, synthetic construct
  source 1..23: mol_type = other RNA
caccgctccacctttgcttagaa

SEQ ID NO: 37 - RNA, 23 nt, synthetic construct
  source 1..23: mol_type = other RNA
aaacttctaagcaaaggtggagc

SEQ ID NO: 38 - RNA, 24 nt, synthetic construct
  source 1..24: mol_type = other RNA
caccggcaagggttggagacccaa

SEQ ID NO: 39 - RNA, 24 nt, synthetic construct
  source 1..24: mol_type = other RNA
aaacttgggtctccaacccttgcc

SEQ ID NO: 40 - RNA, 23 nt, synthetic construct
  source 1..23: mol_type = other RNA
caccgcaagggttggagacccaa

SEQ ID NO: 41 - RNA, 23 nt, synthetic construct
  source 1..23: mol_type = other RNA
aaacttgggtctccaacccttgc

SEQ ID NO: 42 - RNA, 24 nt, synthetic construct
  source 1..24: mol_type = other RNA
caccgccgagacccgggccccatg

SEQ ID NO: 43 - RNA, 24 nt, synthetic construct
  source 1..24: mol_type = other RNA
aaaccatggggcccgggtctcggc

SEQ ID NO: 44 - RNA, 21 nt, synthetic construct
  source 1..21: mol_type = other RNA
caccgagacccgggccccatg

SEQ ID NO: 45 - RNA, 21 nt, synthetic construct
  source 1..21: mol_type = other RNA
aaaccatggggcccgggtctc

SEQ ID NO: 46 - RNA, 25 nt, synthetic construct
  source 1..25: mol_type = other RNA
caccgcccatgtggagcaggatcag

SEQ ID NO: 47 - RNA, 25 nt, synthetic construct
  source 1..25: mol_type = other RNA
aaacctgatcctgctccacatgggc

SEQ ID NO: 48 - RNA, 23 nt, synthetic construct
  source 1..23: mol_type = other RNA
caccgcatgtggagcaggatcag

SEQ ID NO: 49 - RNA, 23 nt, synthetic construct
  source 1..23: mol_type = other RNA
aaacctgatcctgctccacatgc

SEQ ID NO: 50 - RNA, 25 nt, synthetic construct
  source 1..25: mol_type = other RNA
caccgtccccatctctggactcgtc

SEQ ID NO: 51 - RNA, 25 nt, synthetic construct
  source 1..25: mol_type = other RNA
aaacgacgagtccagagatggggac

SEQ ID NO: 52 - RNA, 23 nt, synthetic construct
  source 1..23: mol_type = other RNA
caccgcccatctctggactcgtc

SEQ ID NO: 53 - RNA, 23 nt, synthetic construct
  source 1..23: mol_type = other RNA
aaacgacgagtccagagatgggc

SEQ ID NO: 54 - RNA, 25 nt, synthetic construct
  source 1..25: mol_type = other RNA
caccgtctggactcgtccggccctt

SEQ ID NO: 55 - DNA, 337 bp, Bos taurus
  source 1..337: mol_type = other DNA
tgggccctttaaatcctaagctccacctttgcttagaagggtctttgggaatataaaagggggtgcagttcctctctgctcctgaaaaccatcagctgctcctgtctgcgggcccccagcccacttctctccagccacccaccaccaacactcccccgggtgccatgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcggggg

SEQ ID NO: 56 - DNA, 133 bp, Bos taurus
  source 1..133: mol_type = other DNA
tgggccctttaaatcctaagctccacctttgcttagaagggtctttgggaatataacaagggactggggagccgagacccgggccccatgtggagcaggatcaggggcagggcggacgcggggccggcggggg

SEQ ID NO: 57 - DNA, 120 bp, Bos taurus
  source 1..120: mol_type = other DNA
tgggccctttaaatcctaagctccacctttgcttagaagggtctttgggactggggagccgagacccgggccccatgtggagcaggatcaggggcagggcggacgcggggccggcggggg

SEQ ID NO: 58 - DNA, 113 bp, Bos taurus
  source 1..113: mol_type = other DNA
tgggccctttaaatcctaagctccacctttgcttagaagggtctttgggagccgagacccgggccccatgtggagcaggatcaggggcagggcggacgcggggccggcggggg

SEQ ID NO: 59 - DNA, 129 bp, Bos taurus
  source 1..129: mol_type = other DNA
tgggccctttaaatcctaagctccacctttgcttagaagggtctttgggggtgcagttcctgagccaagtggtgctggcactgatccagagtcgggggcaagggttggagacccaagggactggggagc

SEQ ID NO: 60 - DNA, 103 bp, Bos taurus
  source 1..103: mol_type = other DNA
tgggccctttaaatcctaagctccacctttgcttagaagggtctttgggaatataacatgtggagcaggatcaggggcagggcggacgcggggccggcggggg

SEQ ID NO: 61 - DNA, 104 bp, Bos taurus
  source 1..104: mol_type = other DNA
tgggccctttaaatcctaagctccacctttgcttagaagggtctttgggaatataaacatgtggagcaggatcaggggcagggcggacgcggggccggcggggg

SEQ ID NO: 62 - DNA, 106 bp, Bos taurus
  source 1..106: mol_type = other DNA
tgggccctttaaatcctaagctccacctttgcttagaagggtctttgggagtgcagttccatgtggagcaggatcaggggcagggcggacgcggggccggcggggg

SEQ ID NO: 63 - DNA, 48 bp, Bos taurus
  source 1..48: mol_type = other DNA
tgggccctttaaatcctaagctccacctttgcttagaagggtctaagg

SEQ ID NO: 64 - DNA, 89 bp, Bos taurus
  source 1..89: mol_type = other DNA
tgggccctttaaatcctaagctccacctttgcttagaagggtctttgggaatataatcaggggcagggcggacgcggggccggcggggg

SEQ ID NO: 65 - DNA, 88 bp, Bos taurus
  source 1..88: mol_type = other DNA
tgggccctttaaatcctaagctccacctttgcttagaagggtctttgggaatataacaggggcagggcggacgcggggccggcggggg

SEQ ID NO: 66 - DNA, 45 bp, Bos taurus
  source 1..45: mol_type = other DNA
tgggccctttaaatcctaagctccacctttgcttagaaggggggg

SEQ ID NO: 67 - DNA, 62 bp, Bos taurus
  source 1..62: mol_type = other DNA
tgggccctttaaatcctaagctccacctttgcttagaagggcggacgcggggccggcggggg

SEQ ID NO: 68 - DNA, 133 bp, Bos taurus; identical to SEQ ID NO: 56
  source 1..133: mol_type = other DNA

SEQ ID NO: 69 - DNA, 98 bp, Bos taurus
  source 1..98: mol_type = other DNA
tgggccctttaaatcctaagctccacctttgcttagaagggtctttgggaatatgtggagcaggatcaggggcagggcggacgcggggccggcggggg

SEQ ID NO: 70 - DNA, 103 bp, Bos taurus; identical to SEQ ID NO: 60
  source 1..103: mol_type = other DNA

SEQ ID NO: 71 - DNA, 89 bp, Bos taurus
  source 1..89: mol_type = other DNA
tgggccctttaaatcctaagctccacctttgcttagaagggtctttgggaatatgatcaggggcagggcggacgcggggccggcggggg

SEQ ID NO: 72 - DNA, 90 bp, Bos taurus
  source 1..90: mol_type = other DNA
tgggccctttaaatcctaagctccacctttgcttagaagggtctttgggaagcaggatcaggggcagggcggacgcggggccggcggggg

SEQ ID NO: 73 - DNA, 103 bp, Bos taurus; identical to SEQ ID NO: 60
  source 1..103: mol_type = other DNA

SEQ ID NO: 74 - DNA, 68 bp, Bos taurus
  source 1..68: mol_type = other DNA
tgggccctttaaatcctaagctccacctttgcttagaaggggcagggcggacgcggggccggcggggg

SEQ ID NO: 75 - DNA, 70 bp, Bos taurus
  source 1..70: mol_type = other DNA
tgggccctttaaatcctaagctccacctttgcttagaagggtctttgggcggacgcggggccggcggggg

SEQ ID NO: 76 - DNA, 89 bp, Bos taurus; identical to SEQ ID NO: 64
  source 1..89: mol_type = other DNA

SEQ ID NO: 77 - DNA, 126 bp, Bos taurus
  source 1..126: mol_type = other DNA
tgggccctttaaatcctaagctccacctttgcttagaagtggtgctggcactgatccagagtcgggggcaagggttggagacccaagggactggggagccgagacccgggccccatgtggagcagg

SEQ ID NO: 78 - DNA, 25 bp, synthetic construct
  source 1..25: mol_type = other DNA
aaacaagggccggacgagtccagac

SEQ ID NO: 79 - DNA, 21 bp, synthetic construct
  source 1..21: mol_type = other DNA
caccggactcgtccggccctt

SEQ ID NO: 80 - DNA, 21 bp, synthetic construct
  source 1..21: mol_type = other DNA
aaacaagggccggacgagtcc

SEQ ID NO: 81 - DNA, 25 bp, synthetic construct
  source 1..25: mol_type = other DNA
caccgtctcagtacttgatccctgc

SEQ ID NO: 82 - DNA, 25 bp, synthetic construct
  source 1..25: mol_type = other DNA
aaacgcagggatcaagtactgagac

SEQ ID NO: 83 - DNA, 23 bp, synthetic construct
  source 1..23: mol_type = other DNA
caccgtcagtacttgatccctgc

SEQ ID NO: 84 - DNA, 23 bp, synthetic construct
  source 1..23: mol_type = other DNA
aaacgcagggatcaagtactgac

SEQ ID NO: 85 - RNA, 20 nt, synthetic construct
  source 1..20: mol_type = other RNA
gtagtccttccacatgtcaa

SEQ ID NO: 86 - RNA, 20 nt, synthetic construct
  source 1..20: mol_type = other RNA
agtctctctaccgccgcagt

SEQ ID NO: 87 - DNA, 910 bp, synthetic construct
  source 1..910: mol_type = other DNA
aatggctctggggtgtatggagggagcaggcccccagccctggaacaaggaaggatgggggtgggggcgtttgcagacatgaagttttgttgaaaggacagaaaaactgaggcccagggagacgggtctttccagaggtcacagagcaccttgcaggccaagcaagcaccagagacaacaggaaagacccccttccacatctccatggggaattaaagtcagaaggaatcaaaggtgaggagtgggccctttaaatcctaagctccacctttgcttagaagggtctttgggaatataaaagggggtgcagttcctctctgctcctgaaaaccatcagctgctcctgtctgcgggcccccagcccacttctctccagccacccaccaccaacactcccccgggtgccatgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcaactcagccggccgcaaggtcaagcgctgaagaccgtcaggtacccacccgctgcagccccaaccctccctggttcagccctcccaagcccctggactctccccatctctggactcg

SEQ ID NO: 88 - RNA, 45 nt, synthetic construct
  source 1..45: mol_type = other RNA
  misc_feature 25: note = n (25-45)
gaaattaatacgactcactataggngttttagagctagaaatagg

SEQ ID NO: 89 - DNA, 80 bp, synthetic construct
  source 1..80: mol_type = other DNA
aaaagcaccgactcggtgccactttttcaagttgataacggactagccttattttaacttgctatttctagctctaaaac

SEQ ID NO: 90 - DNA, 393 bp, Bos taurus
  source 1..393: mol_type = genomic DNA
agctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcaactcagccg

SEQ ID NO: 91 - DNA, 369 bp, Bos taurus
  source 1..369: mol_type = other DNA
agctaccatgtggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtgtgtgttgggcgcaactcagccg

SEQ ID NO: 92 - DNA, 337 bp, Bos taurus
  source 1..337: mol_type = other DNA
agctaccatgtggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagagtgggcgcaactcagccg

SEQ ID NO: 93 - DNA, 11 bp, Bos taurus
  source 1..11: mol_type = other DNA
caactcagccg

SEQ ID NO: 94 - DNA, 381 bp, Bos taurus
  source 1..381: mol_type = other DNA
gtaagatgccctttgacattgaaggatacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacacgggtctctcctctgcagtgggcgcaactcagccg

SEQ ID NO: 95 - DNA, 19 bp, Bos taurus
  source 1..19: mol_type = other DNA
agtgggcgcaactcagccg

SEQ ID NO: 96 - DNA, 354 bp, Bos taurus
  source 1..354: mol_type = other DNA
ccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccagtgggcgcaactcagccg

SEQ ID NO: 97 - DNA, 30 bp, Bos taurus
  source 1..30: mol_type = other DNA
agctgccacccagtgggcgcaactcagccg

SEQ ID NO: 98 - DNA, 354 bp, Bos taurus
  source 1..354: mol_type = other DNA
agctgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctacc

SEQ ID NO: 99 - DNA, 417 bp, Bos taurus
  source 1..417: mol_type = other DNA
atgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcaactcagccggccgcaaggtcaagcgctga

SEQ ID NO: 100 - DNA, 395 bp, Bos taurus
  source 1..395: mol_type = genomic DNA
atgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgcagtgggcgcactcaacc

SEQ ID NO: 101 - DNA, 399 bp, Bos taurus
  source 1..399: mol_type = genomic DNA
atgcagctgccaccctttgacatgtggaaggactacttcaacctgagccaagtggtgctggcactgatccagagtcgggggcaagggttggagacccaagggactggggagccgagacccgggccccatgtggagcaggatcaggggcagggcggacgcggggctggcgggggcctggccaccctgtgcaacttttgcaaacacaatggagagtctcgccacgtgtactcctcacaccagctgaagaccccggagggcgtggtggtgtgtcccattctgcggcattatgtatgtcccctgtgcggggccaccggggaccaggcccacacactcaagtactgcccactcaacggaggacagcagtctctctaccgccgctgggcgcactcagccgcgcca